(19) United States
(12) Patent Application Publication
(10) Pub. No.: US 2015/0133918 A1
(43) Pub. Date: May 14, 2015

(54) PERCUTANEOUS CATHETER-BASED ARTERIAL DENERVATION WITH INTEGRAL EMOBOLIC FILTER

(71) Applicant: CONTEGO MEDICAL, LLC, Raleigh, NC (US)

(72) Inventor: RAVISH SACHAR, Raleigh, NC (US)

(21) Appl. No.: 14/537,397

(22) Filed: Nov. 10, 2014

Related U.S. Application Data
(60) Provisional application No. 61/901,734, filed on Nov. 8, 2013.

(51) Int. Cl. A61B 18/14; A61B 17/221

(52) U.S. Cl. CPC A61B 18/1492; A61B 17/221; A61B 2018/00434
USPC 606/41

(57) ABSTRACT

A neuromodulation device having means for selective denervation of nerves in a selected portions of a blood vessel; and an embolic filter mounted to the catheter shaft at a location distal to the catheter balloon. Thus the filter can be down-stream from the blockage and can be properly positioned to capture embolic particles that can be set loose into the blood stream as the neuromodulation procedure can be performed. The embolic filter can be normally un-deployed against the catheter shaft to facilitate introduction and withdrawal of the device to and from the operative site. Once the neuromodulation device is properly positioned, however, means operatively associated with the embolic filter can be actuated to deploy the filter to position a filter mesh across the lumen of the vessel.

### CROSS REFERENCE TO RELATED APPLICATION

This application claims the benefit of U.S. Provisional Application No. 61/901,734, filed Nov. 8, 2013, which is hereby incorporated herein by reference in its entirety.

### BACKGROUND

1. Field of the Invention

Implementations described herein relate generally to surgical devices and relate more specifically to percutaneous devices, systems and methods for neuromodulation with an integral embolic filter.

2. Related Art

It is known that progressively decreasing perfusion of the kidneys is a principal non-cardiac cause perpetuating the downward spiral of congestive heart failure (“CHF”), which is a condition that occurs when the heart becomes damaged and reduces blood flow to the organs of the body. If blood flow decreases sufficiently, kidney function becomes altered, which results in fluid retention, abnormal hormone secretions and increased constriction of blood vessels. These results increase the workload of the heart and further decrease the capacity of the heart to pump blood through the kidneys and circulatory system. Moreover, the fluid overload and associated clinical symptoms resulting from these physiologic changes result in additional hospital admissions, poor quality of life and additional costs to the health care system.

In addition to their role in the progression of CHF, the kidneys play a significant role in the progression of Chronic Renal Failure (“CRF”), End-Stage Renal Disease (“ESRD”), hypertension (pathologically high blood pressure) and other cardio-renal diseases. It has been established in animal models that heart failure typically results in abnormally high sympathetic activation of the kidneys in which an increase in renal sympathetic nerve activity leads to decreased removal of water and sodium from the body, as well as increased renin secretion.

Increased renin secretion leads to vasoconstriction of blood vessels supplying the kidneys which causes decreased renal blood flow. Reduction of sympathetic renal nerve activity, e.g., via denervation, can reverse these processes.

It is further known that the vascular bed of patients suffering from the noted disorders can be damaged and can comprise a range of material from early-stage thrombosis to late-stage calcified plaque. Any procedure, such as neuromodulation, that is used to open up a blocked vessel and restore blood flow or is compressed against the wall of the vessels of the patient can release embolic particles down-stream from the stenosed location. These embolic particles can result in adverse clinical consequences. It has been shown beneficial to trap these embolic particles to prevent them from traveling downstream with blood flow to the capillary bed (e.g., Baim D S, Wahr D, George B, et al., Randomized trial of a distal embolic protection device during percutaneous intervention of saphenous vein aorto-coronary bypass grafts, Circulation 2002; 105:1285-90).

In addition to balloon neuromodulation, stenoses can also be treated with stents and with mechanical atherectomy and thrombectomy devices. These devices can be also prone to releasing embolic particles downstream from the stenosed location.

Systems available today used to catch these embolic particles consist primarily of filter systems or occlusion balloon systems, both built on a guidewire. Typically, a filter scaffolding configured to support a filter membrane is mounted at the distal end of the filter guidewire. The filter scaffolding is movable between a retracted position, in which the scaffolding lies against the guidewire for insertion and retraction of the guidewire in the patient's body, and an expanded position in which the filter medium expands across substantially the entire vessel. In use, the prior art filter guidewire is inserted through the main lumen of the neuromodulation catheter and advanced to a “landing zone” distal to the stenosis. The filter guidewire is then manipulated to deploy a filter scaffolding having a filter medium attached and configured to capture any emboli released by the neuromodulation procedure.

These systems suffer shortcomings related to simplicity of use and crossing tight lesions with a filter or balloon guidewire that can be larger in diameter than the guidewire which would normally be used. These embolic protection guidewires also suffer from flexibility and stability problems that render the protected neuromodulation procedure relatively more difficult in many cases. In the case of saphenous vein grafts, the problems relate specifically to aorto-ostial lesions, where the guidewire cannot be long enough to provide support, or distal vein graft lesions and renal artery lesions, where there can be not enough of a landing zone for the filter. The latter can be a problem as currently available filter systems can have a considerable distance between the treatment balloon and the distal filter. This distance can be a problem not only in distal vein graft lesions, but also in arterial stenoses in which there can be a side branch immediately after the stenosis, such as native coronary arteries. In such cases, the filter can often be deployed only distal to the side branch, thus leaving the side branch unprotected from embolic particles.

Accordingly, a need exists for improved systems and methods of neuromodulation of desired blood vessels to support the efficacious treatment of the patient while minimizing embolic risk to the patient as a result of the treatment..

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Stated generally, the present disclosure comprises a percutaneous transluminal neuromodulation device with an integral embolic filter. Because the filter can be integral with the catheter of the neuromodulation device, any need to insert a separate device into the vessel can be eliminated. Further, proper placement of the neuromodulation balloon can assure proper placement of the embolic filter.

Stated more specifically, the present disclosure comprises a catheter having an elongated shaft, proximal and distal ends, a longitudinal axis and a filter. The filter comprises a first ring coaxially fixedly mounted on a distal portion of the catheter shaft, a second ring coaxially slidably mounted on a distal portion of the catheter shaft and configured to be moved toward and away from the first ring and a scaffolding extending between the first and second rings. The scaffolding further comprises a plurality of first longitudinal connecting members, each having a first end attached to the first ring and a second end extending toward the second ring; a plurality of second longitudinal connecting members, each having a first end attached to the second ring and a second end extending toward the first ring. Each of the first and second longitudinal connecting members further comprise a bifurcation formed on the second end thereof, each of the bifurcations comprising first and second branches; and a means for connecting a branch on each of the plurality of first longitudinal connecting members to a branch on an opposite one of the plurality of second longitudinal connecting members. The filter further comprises a membrane connected to at least the scaffolding.

Means for selective neuromodulation of a desired portion of the patient's nervous system in a selected vessel are described herein. For example, and without limitation, the means for selective neuromodulation can comprise the use of one or more of: neuromodulation via a pulsed electric field (“PEF”), neuromodulation via a stimulation electric field, neuromodulation via localized drug delivery, neuromodulation via high frequency ultrasound, neuromodulation via thermal techniques, and combinations thereof, etc.

Such means for selective neuromodulation can, for example, effectuate irreversible electroporation or electrofusion, necrosis and/or inducement of apoptosis, alteration of gene expression, action potential blockade or attenuation, changes in cytokine up-regulation and other conditions in target neural fibers. In some patients, when the neuromodulatory methods and apparatus described herein are applied to renal nerves and/or other neural fibers that contribute to renal neural functions, the neuromodulatory effects induced by the neuromodulation can result in increased urine output, decreased plasma renin levels, decreased tissue (e.g., kidney) and/or urine catecholamines (e.g., norepinephrine), increased urinary sodium excretion, and/or controlled blood pressure. Furthermore, it is believed that these or other changes might prevent or treat congestive heart failure, hypertension, acute myocardial infarction, end-stage renal disease, contrast nephropathy, other renal system diseases, and/or other renal or cardio-renal anomalies. The methods, systems and apparatus described herein could be used to modulate efferent or afferent nerve signals, as well as combinations of efferent and afferent nerve signals.

Renal neuromodulation preferably is performed in a bilateral fashion, such that neural fibers contributing to renal function of both the right and left kidneys are modulated. Bilateral renal neuromodulation can provide enhanced therapeutic effect in some patients as compared to renal neuromodulation performed unilaterally, i.e., as compared to renal neuromodulation performed on neural tissue innervating a single kidney. In some embodiments, concurrent modulation of neural fibers that contribute to both right and left renal function can be achieved. In additional or alternative embodiments, such modulation of the right and left neural fibers can be sequential. Bilateral renal neuromodulation can be continuous or intermittent, as desired.

When utilizing an electric field for neuromodulation, the electric field parameters can be altered and combined in any combination, as desired. Such parameters can include, but are not limited to, voltage, field strength, pulse width, pulse duration, the shape of the pulse, the number of pulses and/or the interval between pulses (e.g., duty cycle), etc. For example and without limitation, when utilizing a pulsed electric field, suitable field strengths can be up to about 10,000 V/cm and suitable pulse widths can be up to about 1 second. In another aspect, and for example and without limitation, suitable shapes of the pulse waveform include, for example, AC waveforms, sinusoidal waves, cosine waves, combinations of sine and cosine waves, DC waveforms, DC-shifted AC waveforms, RF waveforms, square waves, trapezoidal waves, exponentially-decaying waves, or combinations. The field includes at least one pulse, and in many applications the field includes a plurality of pulses. Suitable pulse intervals include, for example, intervals less than about 10 seconds.

Additional features and advantages of exemplary implementations of the disclosure will be set forth in the description which follows, and in part will be obvious from the description, or can be learned by the practice of such exemplary implementations. The features and advantages of such implementations can be realized and obtained by means of the instruments and combinations particularly pointed out in the appended claims. These and other features will become more fully apparent from the following description and appended claims, or can be learned by the practice of such exemplary implementations as set forth hereinafter.

### BRIEF DESCRIPTION OF THE DRAWINGS

The accompanying drawings, which are incorporated in and constitute a part of this specification, illustrate aspects and together with the description, serve to explain the principles of the methods and systems.

FIG. 1 illustrates a side view of one aspect of a neuromodulation device with integral embolic filter.

FIG. 2A illustrates a cross-section of the proximal end of the neuromodulation device with integral embolic filter shown in FIG. 1; and FIG. 2B illustrates a cross-section of the distal end of the device shown in FIG. 1.

FIG. 3 illustrates a schematic view of one aspect of a filter scaffolding of the neuromodulation device of FIG. 1, showing the filter scaffolding in an un-deployed position.

FIG. 4 illustrates a schematic view of the filter scaffolding of FIG. 3, showing the filter scaffolding in a deployed position.

FIG. 5 illustrates a schematic view of another aspect of a filter scaffolding of the neuromodulation device of FIG. 1, showing the filter scaffolding in an un-deployed position.

FIG. 6 illustrates a schematic view of the filter scaffolding of FIG. 5, showing the filter scaffolding in a deployed position.

FIG. 7 illustrates a schematic view of a third aspect of a filter scaffolding of the neuromodulation device of FIG. 1, showing the filter scaffolding in an un-deployed position.

FIG. 8 illustrates a schematic view of the filter scaffolding of FIG. 7, showing the filter scaffolding in a deployed position.

FIG. 9 illustrates a blood vessel having a stenosis.

FIG. 10 illustrates the blood vessel with stenosis of FIG. 9 with the neuromodulation device of FIG. 1 positioned therein.

FIG. 11 illustrates the blood vessel and neuromodulation device of FIG. 10 with the integral embolic filter expanded.

FIG. 12 illustrates the blood vessel and neuromodulation device of FIG. 10 with the neuromodulation balloon and integral embolic filter deployed.

FIG. 13 illustrates the blood vessel and neuromodulation device of FIG. 10 after treatment of the stenosis, with the neuromodulation balloon in its un-deployed position and the embolic filter still in its deployed position.

FIG. 14 illustrates the blood vessel and neuromodulation device of FIG. 10 after treatment of the stenosis, with both the neuromodulation balloon and embolic filter in an un-deployed position in preparation for withdrawal of the device from the vessel.

FIG. 15 illustrates an alternate aspect of a neuromodulation device having a collapsible filter scaffolding in its un-deployed position.

FIG. 16 illustrates the neuromodulation device of FIG. 15 in its deployed position.

FIG. 17 illustrates a side view of another aspect of a neuromodulation device with integral embolic filter where the treatment device lies distal to the filter.

FIG. 18 illustrates one aspect of a braided wire filter scaffolding that is generally cylindrical shown unrolled and flattened.

FIG. 19 illustrates the braided wire filter scaffolding of FIG. 18 shown stretched out over a mandrel to form a generally cylindrical shape.

FIG. 20 illustrates the braided wire filter scaffolding of FIG. 18 showing the respective ends trimmed to form a desired elongate length.

FIG. 21 illustrates the braided wire filter scaffolding of FIG. 20 that has been heat treated to set the shape memory in the baseline closed position upon application of axial compression.

FIG. 22 is a schematic view illustrating human renal anatomy.

FIG. 23 is a schematic isometric detail view showing the location of the renal nerves relative to the renal artery.

FIGS. 24A and 24B are schematic isometric and end views, respectively, illustrating orienting of an electric field for selectively affecting renal nerves.

FIG. 25 is a schematic side view, partially in section, illustrating an example of an extravascular method and apparatus for renal neuromodulation.

FIGS. 26A and 26B are schematic side views, partially in section, illustrating examples of, respectively, intravascular and intra-to-extravascular methods and apparatus for renal neuromodulation.

FIGS. 27A-27H are schematic side views, partially in section, illustrating methods of achieving bilateral renal neuromodulation utilizing apparatus of the present invention, illustratively utilizing the apparatus of FIG. 26A.

FIGS. 28A and 28B are schematic side views, partially in section, illustrating methods of achieving concurrent bilateral renal neuromodulation utilizing embodiments of the apparatus of FIG. 26A.

FIG. 29 is a schematic side view, partially in section, illustrating methods of achieving concurrent bilateral renal neuromodulation utilizing an alternative embodiment of the apparatus of FIG. 25.

FIG. 30 is a schematic view illustrating an example of methods and apparatus for achieving bilateral renal neuromodulation via localized drug delivery.

### DETAILED DESCRIPTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention provided as an enabling teaching of the invention in its best, currently known aspect. To this end, those skilled in the relevant art will recognize and appreciate that many changes can be made to the various aspects of the invention described herein, while still obtaining the beneficial results described herein. It will also be apparent that some of the desired benefits described herein can be obtained by selecting some of the features described herein without utilizing other features. Accordingly, those who work in the art will recognize that many modifications and adaptations to the present invention are possible and can even be desirable in certain circumstances and are a part described herein. Thus, the following description is provided as illustrative of the principles described herein and not in limitation thereof.

Reference will be made to the drawings to describe various aspects of one or more implementations of the invention. It is to be understood that the drawings are diagrammatic and schematic representations of one or more implementations, and are not limiting of the present disclosure. Moreover, while various drawings are provided at a scale that is considered functional for one or more implementations, the drawings are not necessarily drawn to scale for all contemplated implementations. The drawings thus represent an exemplary scale, but no inference should be drawn from the drawings as to any required scale.

In the following description, numerous specific details are set forth in order to provide a thorough understanding described herein. It will be obvious, however, to one skilled in the art that the present disclosure can be practiced without these specific details. In other instances, well-known aspects of percutaneous transluminal neuromodulation devices and embolic filters have not been described in particular detail in order to avoid unnecessarily obscuring aspects of the disclosed implementations.

As used in the specification and the appended claims, the singular forms “a,” “an” and “the” include plural referents unless the context clearly dictates otherwise. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance can or cannot occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The term “ablate” may refer to the act of altering a tissue to suppress or inhibit its biological function or ability to respond to stimulation. For example and without limitation, ablation may involve, but is not limited to, thermal necrosis (e.g. using energy such as thermal energy, radiofrequency electrical current, direct current, microwave, ultrasound, high intensity focused ultrasound, and laser), cryogenic ablation, electroporation, selective devernation (e.g. destruction of desired nerves at the treatment location, such as, for example and without limitation, destruction of desired afferent nerves from the carotid body while preserving nerves from the carotid sinus which conduct baroreceptor signals), embolization (e.g. occlusion of blood vessels feeding the gland), artificial sclerosing of blood vessels, mechanical impingement or crushing, surgical removal, chemical ablation, or application of radiation causing controlled necrosis (e.g. brachytherapy). In one aspect, the system and methods described herein can be configured to involve inserting a catheter in the patient's vascular system, positioning an energy delivery element thereron the catheter proximate to desired location within the patient's blood vessels and delivering ablative thermal energy to the desire nerves in order to ablate them.

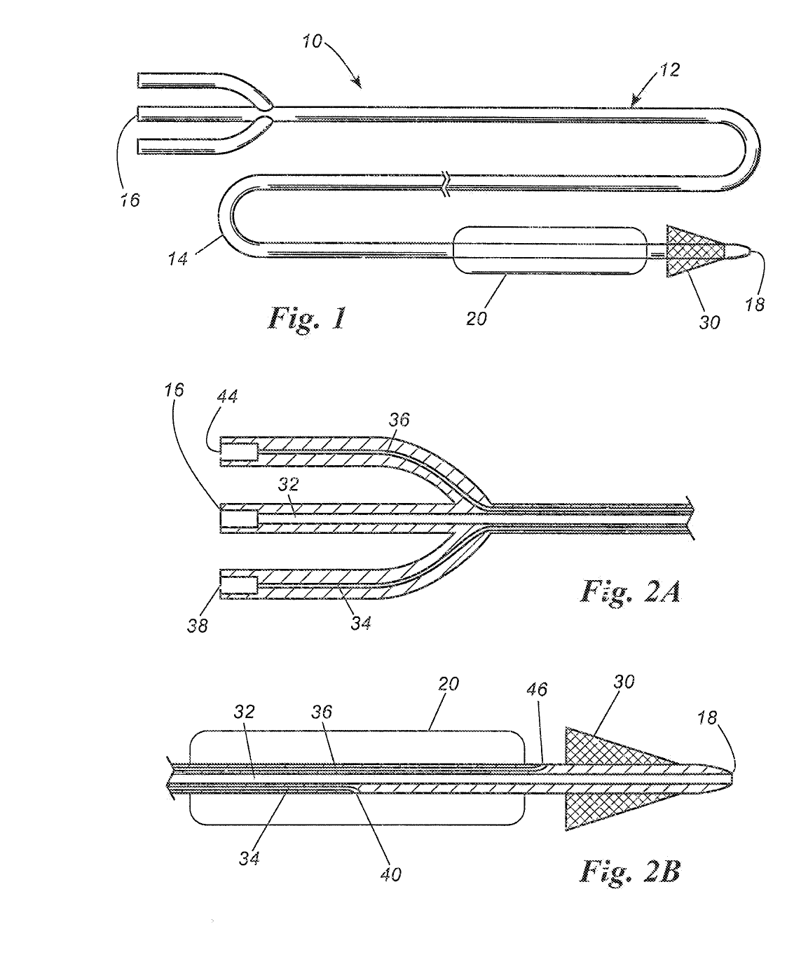

Referring now to the drawings, in which identical numbers indicate identical elements throughout the various views, FIG. 1 illustrates a first aspect of a neuromodulation catheter with integral embolic filter 10 according to the present invention. The neuromodulation catheter with integral embolic filter 10 comprises an elongated catheter 12 having a shaft 14 with a proximal end 16 and a distal end 18. As used herein, “proximal” refers to the portion of the device closest to the physician performing the procedure and “distal” refers to the portion of the device that is furthest from the physician performing the procedure. A neuromodulation treatment device 20 can be mounted to the catheter 12 at a location near the distal end 18 of the catheter shaft 14. Neuromodulation treatment devices can comprise, for example and without limitation, balloons, stents, wires, and multiple types of scaffolds and energy delivery devices that can deliver therapy for effecting neuromodulation, e.g., denervation, operatively coupled thereto.

The methods, systems and apparatus for providing the means for effecting neuromodulation can be used to achieve bilateral renal neuromodulation. In some embodiments, concurrent modulation of neural fibers that contribute to both right and left renal function may be achieved. In additional or alternative embodiments, such modulation of the right and left neural fibers may be sequential. Bilateral renal neuromodulation may be continuous or intermittent, as desired.

The means for effecting neuromodulation can be used to modulate neural fibers that contribute to renal function and may exploit any suitable neuromodulatory techniques that will achieve the desired neuromodulation. For example, any suitable electrical signal or field parameters, e.g., any electric field that will achieve the desired neuromodulation (e.g., electroporative effect) may be utilized. Alternatively or additionally, neuromodulation may be achieved via localized delivery of a neuromodulatory agent or drug.

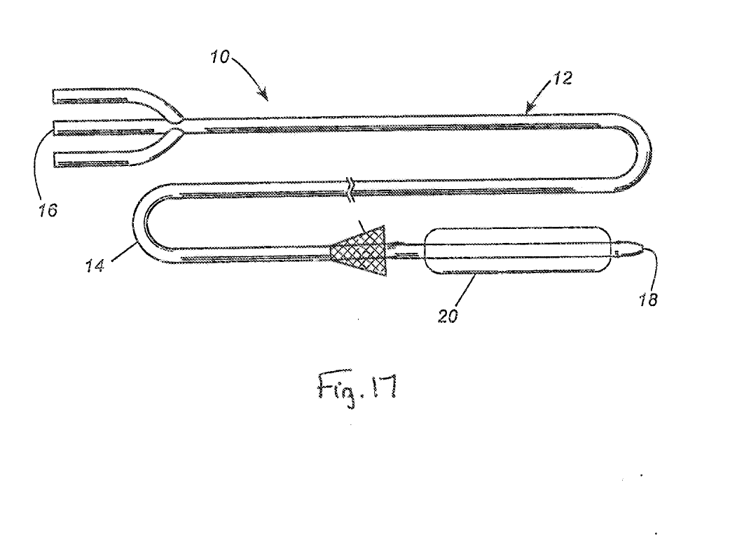

An embolic filter 30 can be mounted to the catheter shaft 14 at a location distal to the neuromodulation treatment device 20 and at or proximal to the distal end 18 of the catheter 12. As illustrated in FIG. 17, it is also contemplated that the embolic filter 30 can be mounted to the catheter shaft 14 at a location proximal to the treatment device 20. In additional or alternative embodiments, the filter 30 can be oriented to face towards or away from the treatment device. One skilled in the art will also appreciate in light of the present disclosure that the neuromodulation catheter can be configured to be, for example and without limitation, an over-the-wire catheter, a rapid-exchange catheter and the like. It is solely for clarity of disclosure that the present description describes an over-the-wire catheter modality.

Referring now to FIG. 2, the catheter shaft 14 can define three lumens: a main lumen 32, a neuromodulation balloon inflation lumen 34, and an embolic filter actuator wire lumen 36. The main lumen 32 can extend from the proximal end 16 to the distal end 18 of the catheter shaft 14. The main lumen 32 can optionally provide a working channel and be configured to receive a guidewire therethrough for advancing the distal end 18 of the catheter 12 through the patient's vasculature to a treatment site. As used herein, the term “treatment site” refers to the location of the occlusion within the patient's vasculature, and when the catheter 12 is referred to as being located or positioned at the treatment site, it will be understood to mean that the catheter is positioned such that the neuromodulation treatment device 20 is located within the occlusion.

The balloon inflation lumen 34 can extend from a proximal port 38 at the proximal end 16 of the catheter 12 and through the catheter shaft 14 to a distal port 40 located within the neuromodulation treatment device 20. Similarly, the actuator wire lumen 36 can extend from a proximal port 44 at the proximal end 16 of the catheter 12 and through the catheter shaft 14 to a distal port 46 distal to the neuromodulation treatment device 20.

Unless otherwise stated, all of the aspects disclosed below share the foregoing characteristics, and the various aspects differ primarily in the design of the embolic filter. Thus, as the various aspects are disclosed, it will be understood unless stated otherwise that each aspect includes the foregoing features, and the description will instead focus on the design and operation of the embolic filter.

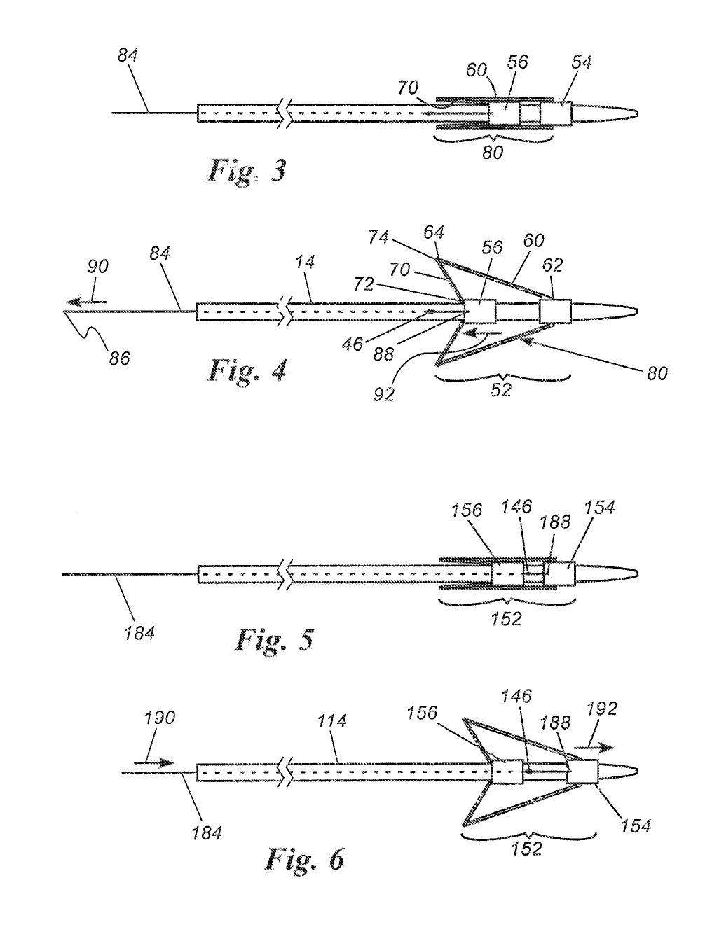

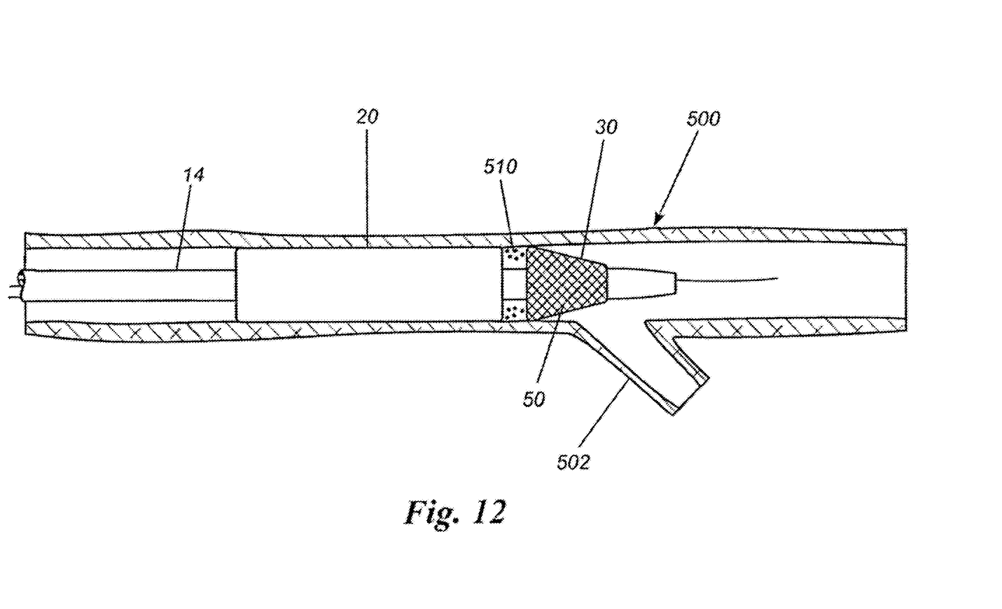

Referring to aspects of the present disclosure illustrated in FIGS. 3 and 4, the embolic filter 30 comprises a filter membrane 50 (FIG. 12) having holes selectively sized to permit the passage of blood but to capture particles larger than normal blood particles and a collapsible scaffolding 52 for supporting the filter membrane. For clarity of illustration, the drawing figures omit the filter membrane 50 when illustrating the scaffolding 52, but it will be understood that all embolic filters disclosed in this application comprise a filter membrane supported by the scaffolding. It is contemplated that the scaffolding 52 can include a proximal ring 56 and a distal ring 54. In one aspect, both of the rings can be located between the distal end of the neuromodulation treatment device 20 and the distal end 18 of the catheter shaft. In a further aspect, the distal ring 54 can be fixed in place on the catheter shaft 14, and the proximal ring 56 can be slidably mounted to the catheter shaft for axial movement in the proximal and distal directions.

Each of a plurality of first strut sections 60 can have a first end 62 and a second end 64. The first end 62 of each first strut section 60 can be attached to the distal ring 54, and each first strut section can extend in the proximal direction.

In other aspects, each of a corresponding plurality of second strut sections 70 can have a first end 72 and a second end 74. Here, the first end 72 of each second strut section 70 can be attached to the proximal ring 56, and each second strut section can also extend in the proximal direction.

In yet other aspects, the second end 64 of each first strut section 60 can attach to the second end 74 of a corresponding second strut section 70. Here, each connected first and second strut section 60, 70 collectively comprises a strut 80. As one skilled in the art will appreciate from the discussion supra, a plurality of strut 80 can be spaced circumferentially about and connecting the proximal and distal rings to form the scaffolding 52. In operation and as shown in FIG. 3, when the proximal and distal rings 56, 54 are adjacent one another each strut 80 can be configured to fold back upon itself. Additionally, when the proximal ring 56 is proximally displaced from the distal ring 54, the struts 80 can be configured to open in a manner similar to an umbrella. The filter membrane 50 can be supported on the first strut sections 60 such that when the scaffolding 52 opens, as shown in FIG. 4, the filter membrane can deploy in a manner similar to an umbrella canopy.

It is contemplated that each strut can further comprise at least one “zone of weakness,” i.e., a zone of the strut that can be configured to be physically weaker than the majority of the strut in order to control the locations at which the struts bend. One skilled in the art will appreciate that the at least one zone of weakness can be formed in any of a number of ways. In one aspect, a notch can be formed in one or both sides of the strut. In another aspect, at least one of the upper surface and lower surface of the strut can be scored. In another aspect, the at least one zone of weakness can be formed of a material that can be structurally weaker than the material comprising the remainder of the strut. In yet other aspects, the at least one zone of weakness can comprise mechanical hinges. In yet other aspects and as shown in FIG. 15, the apices of the sinusoidal ring 55 comprise a zone of weakness. In even further aspects, at least two of these approaches can be combined to form the at least one zone of weakness, e.g., both notching the width and scoring the depth of the strut. In addition, the at least one zone of weakness can comprise a plurality of one type of physical arrangement, e.g., a single zone of weakness can comprise a plurality of notches or a plurality of scores. In operation, the at least one zone of weakness can be configured to bend the strut in response to a force at a predetermined angle to the longitudinal axis of that portion of the strut.

In operation, movement of the proximal ring 56 toward and away from the distal ring 54 to open and to close the embolic filter 30 can be accomplished by manipulation of an actuator wire 84. In one aspect, the proximal end 86 of the actuator wire 84 can extend out of the proximal port 44 of the actuator wire lumen 36 so as to be controllable by the physician performing the procedure. Here, the actuator wire 84 can extend through the actuator wire lumen 36 and can exit through the distal port 46 of the actuator wire lumen. In another aspect, the distal end 88 of the actuator wire 84 can be attached to the proximal ring 56.

One skilled in the art will appreciate here are a variety of ways in which the filter scaffolding 52 and actuator wire 84 can be arranged to permit the embolic filter 30 to be opened and closed by moving the proximal end 86 of the actuator wire. In a first aspect, the filter scaffolding 52 can be formed in a normally closed or undeployed position. In operation, pulling the proximal end 86 of the actuator wire 84 can cause the proximal ring 56 to slide in a proximal direction to open the filter scaffolding 52. The filter scaffolding can be configured so that releasing the tension on the actuator wire 84 and/or pushing the actuator wire 84 distally can permit the filter scaffolding 52 to collapse to an un-deployed position.

In another aspect of the present disclosure illustrated in FIGS. 5 and 6, a filter scaffolding 152 can comprise a proximal ring 156 that can be fixed with respect to a catheter shaft 114 and a distal ring 154 that can be slidably positioned along the catheter shaft in the proximal and distal directions. In a further aspect, a distal port 146 of an actuator wire lumen 136 can be located distal to the proximal ring 156. Here, an actuator wire (not shown) can extend through the actuator wire lumen, can exit through a distal port 146, and can attach to the distal ring 154. The filter scaffolding 152 can be formed in a normally closed position. In operation, pushing the actuator wire 184 can displace the distal ring 154 in a distal direction away from the proximal ring 156 to deploy the filter scaffolding 152. The filter scaffolding can be configured so that releasing the force on the actuator wire 184 and/or pushing the actuator wire 184 distally can permit the filter scaffolding 152 to return to its un-deployed position.

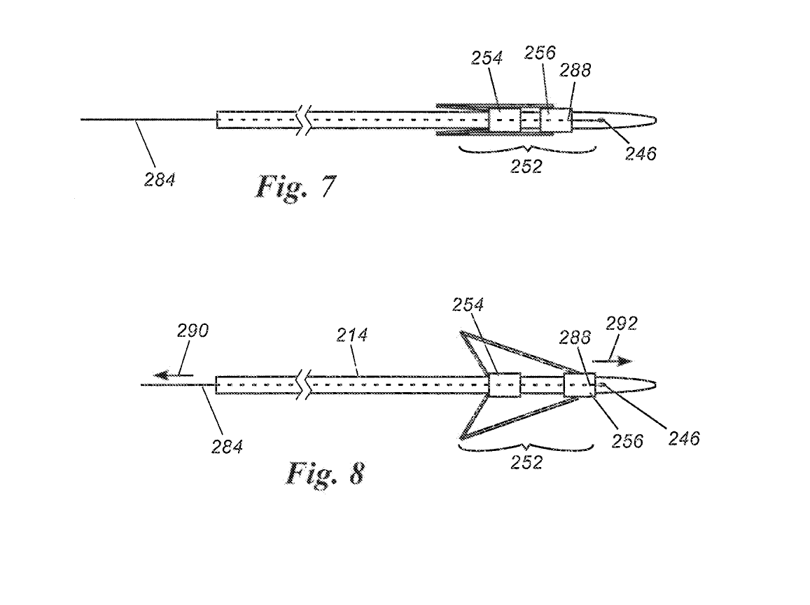

In yet another aspect of the present disclosure illustrated in FIGS. 7 and 8, a proximal ring 254 can be fixed with respect to a catheter shaft 214, and a distal ring 256 can be slidably positioned along the catheter shaft in the proximal and distal directions. In a further aspect, a distal port 246 of an actuator wire lumen 236 can be located distal to the distal ring 256. Here, an actuator wire 284 can extend through the actuator wire lumen 236, can exit through the distal port 246, and can attach to the distal ring 256. The filter scaffolding 252 can be formed in a normally closed position. In operation, pulling on the actuator wire 284 can displace the distal ring 256 in a distal direction and away from the proximal ring 156 to deploy the filter scaffolding 252. The filter scaffolding can be configured so that releasing the force on the actuator wire 284 can permit the filter scaffolding 252 to return to its un-deployed position.

Referring back to FIGS. 3 and 4, another aspect of a filter scaffolding can be structurally identical to the first embodiment 52 except that the filter scaffolding can be formed in a normally open or deployed position. Here, it is contemplated that application of a distally directed force to the proximal end 86 of the actuator wire 84 (i.e., pushing the actuator wire) can maintain the proximal ring 56 in its distal position and hence can maintain the filter scaffolding 52 in its un-deployed position. The filter scaffolding 52 can be permitted to expand to its normally deployed position, expanding the filter membrane 50, upon release of the force applied to the actuator wire 84. Immediately after completion of the interventional procedure, a distally directed force can again be applied to the proximal end 86 of the actuator wire 84, moving the proximal ring 56 toward the distal ring 54 and collapsing the filter scaffolding 52.

Referring back to FIGS. 5 and 6, a fifth aspect can be structurally identical to the third aspect with the exception that the filter scaffolding 152 can be formed in a normally open position. Here, it is contemplated that the distal ring 154 can be normally displaced toward the distal end 18 of the catheter shaft 114. In operation, pulling on the distal end 188 of the actuator wire 184 can move the distal ring 154 proximally toward the fixed proximal ring 156, collapsing the filter scaffolding 152 while releasing the tension on the actuator wire 184 can permit the filter scaffolding 152 to expand to its deployed position.

In those aspects in which the force applied to the actuator wire is configured to be an axial compressive force, those skilled in the art can appreciate that a stiffer wire can be used to prevent buckling of the actuator wire than in those embodiments where the force applied to the actuator wire is configured to be an axial tensile force.

In the present disclosure, and especially in the case of actuator wires, the term “wire” is intended to comprise, for example and without limitation, metallic wires, polymeric wires, and the like. In the case of polymeric wires, the polymers used can comprise, for example and without limitation, nylon, polypropylene and the like.

In the foregoing aspects, the filter membrane 50 can be formed from at least one of a textile, a polymer and a wire mesh. In another aspect, the filter membrane 50 comprises pores and, in a further aspect, the pores can be sized to allow blood to pass but not embolic particles. It is also contemplated that the filter membrane 50 can be mounted either on top of or inside of the frame.

In the foregoing aspects, the filter membrane 50 can be configured to cover the exterior surface of the outermost strut sections, i.e., the first strut sections 60, 160, and 260. Optionally, the filter membrane 50 can be further configured to extend beyond the distal or second ends 64, 164, and 264 of the first strut sections 60, 160, and 260, where it can be attached to the circumference of the distal ring 54, 156, 256. In those aspects in which the distal ring 54 can be fixed, the filter membrane 50 can optionally be configured to extend beyond the distal end of the distal ring and can be attached to the circumference of the catheter shaft 14 at a location between the distal ring 54 and the distal end 18 of the catheter shaft.

It is also contemplated that the filter membrane 50 in each of the disclosed embodiments can be attached to the inner surfaces of the first strut sections 60, 160, and 260 instead of to the outer surfaces.

It is further contemplated that the inner or second strut sections 70, 170, 270 can also be configured in a concave shape with respect to the blood flow when the filter scaffolding is deployed. In further or additional aspects, the filter membrane 50 can be attached to the inner or outer surfaces of the second strut sections 70, 170, 270. When the filter membrane 50 is attached to the surfaces of the second strut sections 70, 170, 270, the filter membrane 50 can optionally extend beyond the distal or second ends 74, 174, 274 of the second strut sections and be attached to the circumference of the proximal ring 56, 154, 254. It is also contemplated that, if the filter membrane 50 can be attached to the outer surfaces of the second strut sections 70 and the proximal ring 56 can be fixed, the filter membrane can be configured to extend beyond the distal end of the proximal ring and can be attached to the catheter shaft 14 at a location between the proximal and distal rings 56, 54.

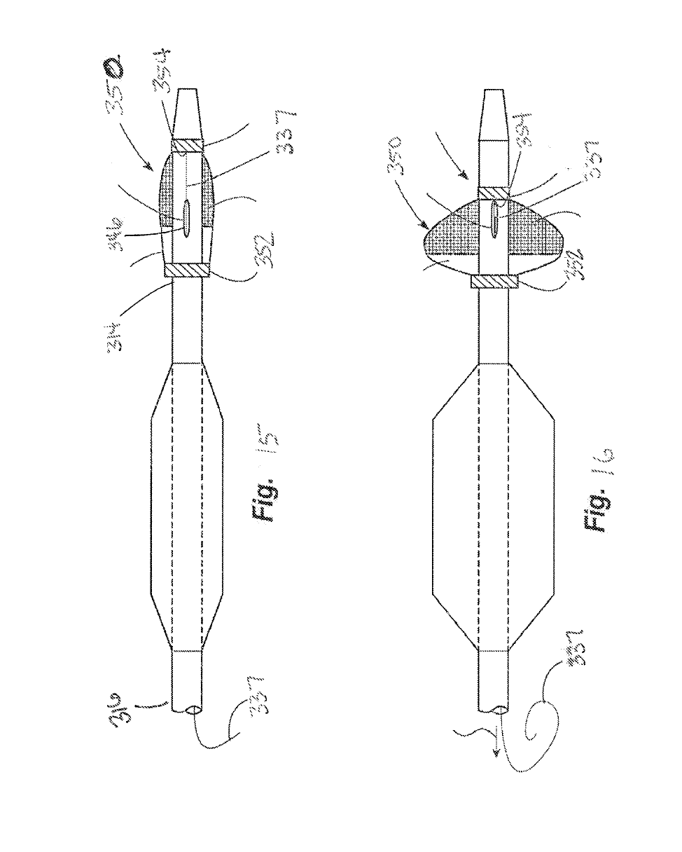

In an alternate aspect shown in FIGS. 15-16, a collapsible filter scaffolding 350. Here, the collapsible filter scaffolding is coupled to a proximal ring 352 and a distal ring 354. In one aspect, it is contemplated that the proximal ring can be fixed in place on the catheter shaft and the distal ring can be slidably mounted to the catheter shaft for axial movement in the proximal and distal directions. It is further contemplated that the actuator wire lumen can extend from a proximal port at the proximal end 316 of the catheter and through the catheter shaft 314 to a distal port 346 located between the proximal and distal rings. A pull wire 337 can extend from the proximal end 316 of the catheter through the distal port 346 and be coupled to the distal ring 354.

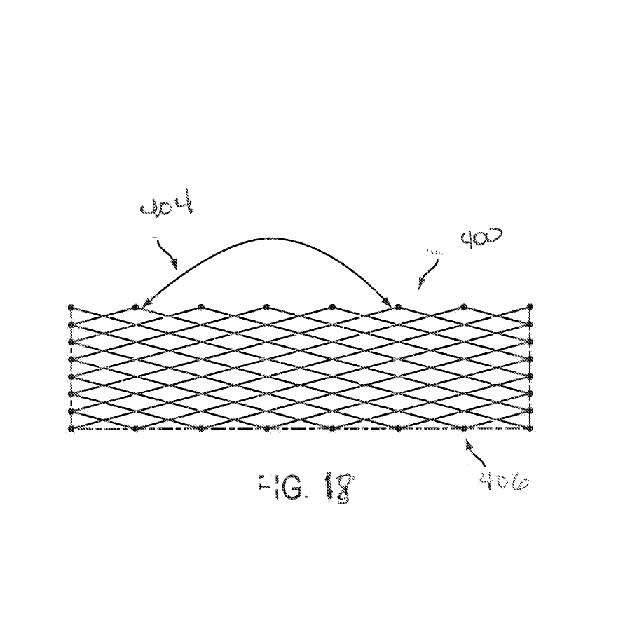

It is further contemplated that the filter scaffolding can be formed from braided wires to form a braided filter scaffolding. FIG. 18 shows a braided wire filter scaffolding 400 that is unrolled and flattened. In this aspect, it will be appreciated that the braided filter scaffolding has a generally cylindrical shape when formed. FIG. 19 illustrates the unformed braided filter scaffolding 400 stretched out over a mandrel to form a substantially cylindrical shape and FIG. 20 shows the respective ends of the formed braided filter scaffolding 400 being trimmed to form a desired elongate longitudinal length. In various optional aspects, it is contemplated that the braided filter scaffolding can be head treated to set the shape memory wires of the braid in the un-deployed or collapsed position. (In an alternative embodiment, the memory wires of the braided filter scaffolding can be heat treated to set the shape memory wires of the brain in the deployed configuration. In this aspect, is it further contemplated that the proximal and distal rings be capable of controlled displacement away from one another, as is discussed elsewhere in the present disclosure.)

With respect to the braided filter scaffolding, the wires can comprise, for example and without limitation, metal wires, polymer wires, and the like. In one aspect, the braided filter scaffolding can be formed from a wire braid comprising from about 12 to about 16 wires. In another aspect, the braided filter scaffolding un-deployed diameter 402 can be from about 0.8 to about 1.0 mm and can be adapted to slidingly fit the catheter shaft diameter. The lead angle between the wires comprising the wire mesh from can be selected to be relatively low to allow the braided filter scaffolding 400 to open to a relatively high diameter when deployed. This deployed diameter 406 can be from about 4 to about 7 mm. The wires comprising the braided filter scaffolding can have a rounded profile in cross-section. The wires comprising the braided filter scaffolding can also be from about 0.002″ to about 0.003″ in diameter or, alternatively the wires can be flat. If the wires are selected to be flat, the wire can be further configured to be about 0.001″×0.003″ in cross-section in order to reduce the profile of the braided filter scaffolding.

In one embodiment, the braided filter scaffolding 400 can be formed from 14 Nitinol or Cobalt-Chromium round wires having a 60 micron diameter and a braiding angle 404 of about 150 degrees on a 7 mm shaft that corresponds to the maximum deployed diameter. In this aspect, the braiding angle can be defined as double (2×) the angle between the wire and the central axis. Optionally, it is contemplated that the braiding angle can be between about 1.5× and 4× or be between 1.7× and 3×. In this aspect, it is contemplated that the braided filter scaffolding 400 can then be compressed to un-deployed diameter 402 of about a 1 mm and heat treated to shape set the form, i.e., to set the base or unstrained shape memory in a base line closed position. Of course, it is also contemplated that the braided filter scaffolding 400 can be heat treated to set the base or unstrained shape memory in a base line open position. It is contemplated that the braided filter scaffolding can form a relatively wide mesh when opened in order to allow blood flow into the filter membrane. It is also contemplated that the braided filter scaffolding can comprise less than 12 wires or more than 16 wires, depending on the desired inhibition or lack thereof to the flow of blood.

It is contemplated that a braided filter scaffolding 400 can be incorporated into the device by joining the distal end 408 of the braided filter scaffolding to a distal ring. In one aspect, the distal end of the braided filter scaffolding can be thermally bonded to a polymer or metallic distal ring. The distal ring can be adapted to slideably fit over the catheter shaft. The proximal end 410 of the braided filter scaffolding can be attached to the proximal ring by thermal bonding as described above or other methods known to one skilled in the art. The distal and proximal rings can be of any length and diameter, but in one aspect, both the proximal and distal ends can have a length from about 0.5 to about 2.0 mm.

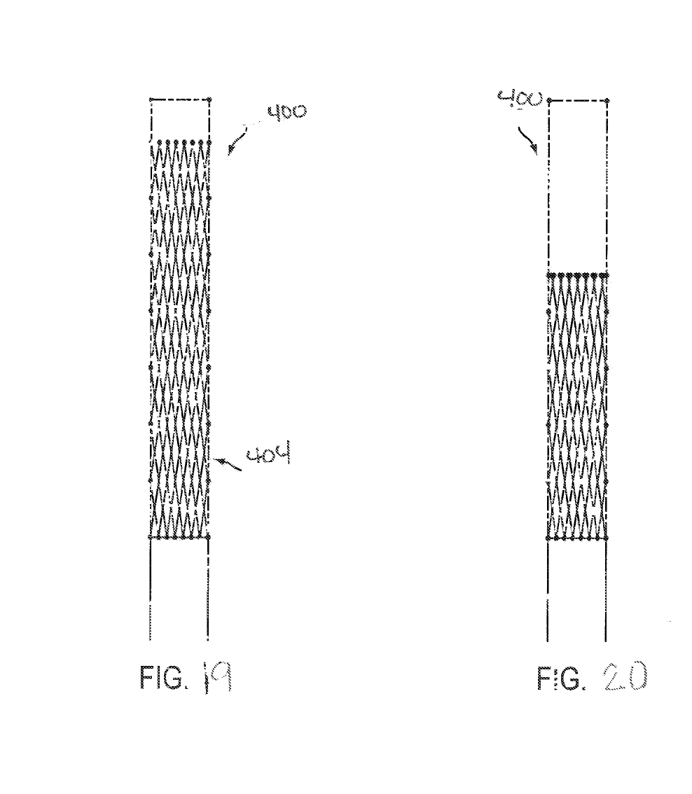

In one exemplary aspect, FIG. 21 shows an exemplary trimmed cylindrically shaped frame design that has been heat treated to set the shape memory in the baseline closed position upon application of an axial compression. For example and not meant to be limiting, the applied axial compression can be exerted by an external operator pulling on a pull wire to controllably cause the mid-portion of the trimmed cylindrically shaped frame design to expand to a desired diameter as the distance between the distal and proximal rings decreases. It is contemplated that this desired diameter can be a multiple of the original diameter of the frame design in the baseline closed position.

In all of the foregoing instances, the filter scaffolding comprises a fixed ring and a movable ring, whereby raising the filter can be accomplished by moving the rings either apart or together, and collapsing the filter can be achieved by moving the rings either together or apart, respectively. “Moving apart” and “moving together” are used as relative terms, such that only one of the two rings need move with respect to the other ring for the rings to “move apart” or “move together.”

Similarly, the process of raising and collapsing the filter can be thought of as being viewed from the perspective of the catheter, such that a movable ring can be moved toward or away from a fixed ring.

In all of the foregoing instances, one can appreciate that both actively applying a force to move a ring and releasing a force to permit the ring to move of its own accord comprise a step of “causing” the movable ring to move by “controlling” the actuator wire. Thus, in both the normally deployed and normally un-deployed filter scaffolding embodiments described herein, the actuator wire can be “controlled” to “cause” a movable ring to move, whether that control takes the form of applying or releasing a force on the actuator wire.

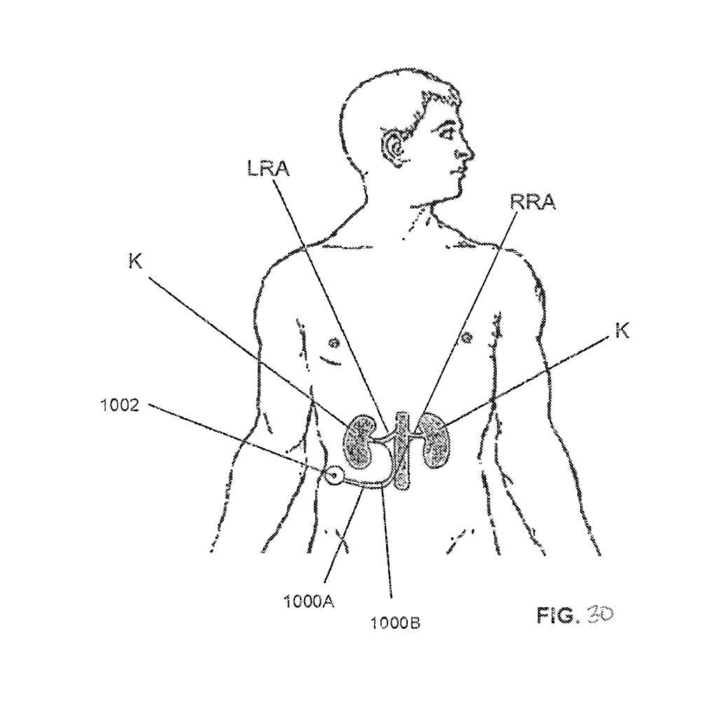

It is also contemplated that, rather than having the physician directly grasp the proximal end of the actuator wire, a control device can be associated with the proximal end of the actuator wire at the proximal end of the catheter shaft. The control device can incorporate, for example and without limitation, levers, sliders, rotating spindles, or the like to facilitate movement of the wire. One example of such a mechanical arrangement is described in U.S. Patent Publication No. US 2010/0106182, paragraphs [0079]-[0090] and FIGS. 29-33, which disclosure is hereby incorporated by reference.

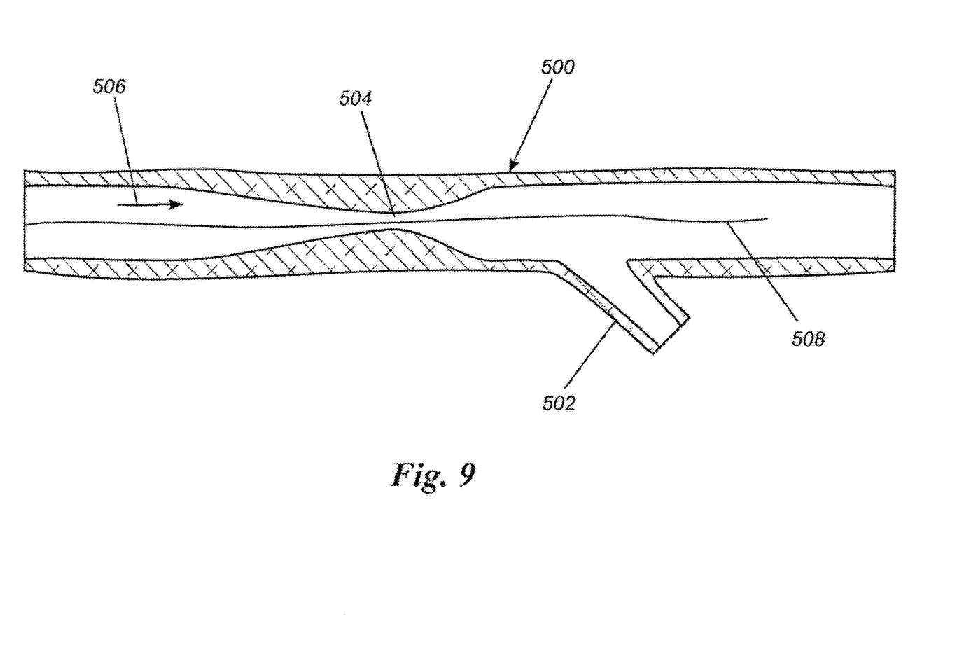

Use of the neuromodulation device with integral embolic filter described above to treat a stenosis in a blood vessel can be shown in FIGS. 9-13. In FIG. 9, a vessel 500 can have a branch vessel 502 diverging from it. The vessel 500 can have a stenosis 504. The direction of blood flow through the vessel 500 is indicated by the arrow 506. A guide wire 508 has been inserted by the physician as a preliminary step in the interventional procedure.

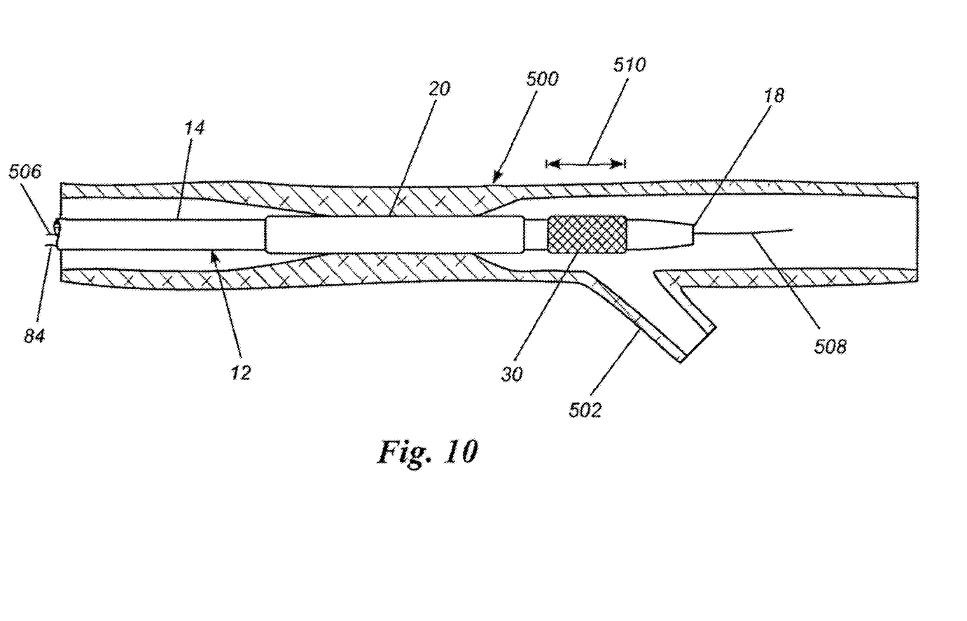

FIG. 10 shows the catheter 12 with neuromodulation balloon 20 and embolic filter 30 in their un-deployed positions and lying adjacent to the catheter shaft 14. The distal end 18 of the catheter shaft 14 has been advanced over the guide wire 506 until the deflated neuromodulation balloon 20 resides within the stenosis. With the catheter 12 positioned such that the neuromodulation balloon 20 can be located within the stenosis, the catheter can be said to be at its “target site.” With the catheter at the target site, the portion of the vessel 500 occupied by the embolic filter 30 can be referred to as the “landing zone” 510.

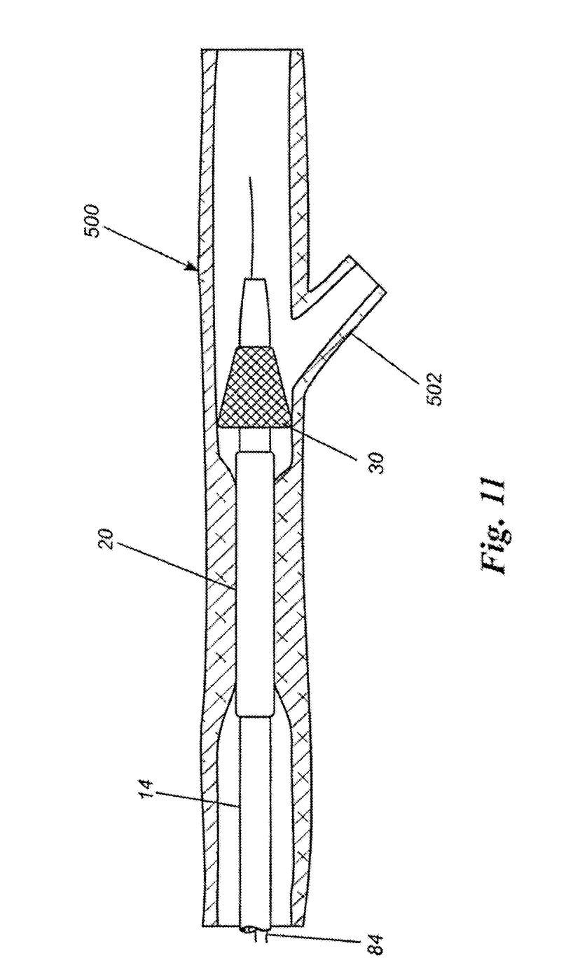

In FIG. 11 the embolic filter 30 has been expanded by pulling on the actuator wire 84. In FIG. 12 the neuromodulation balloon 20 can be inflated and, if needed, deflated and re-inflated, optionally multiple times, to force the stenosis open. In the process of crushing the plaque that forms the stenosis, embolic particles 510 are released and swept by the blood flow into the open proximal end of the embolic filter 30, where they are captured by the filter membrane 50.

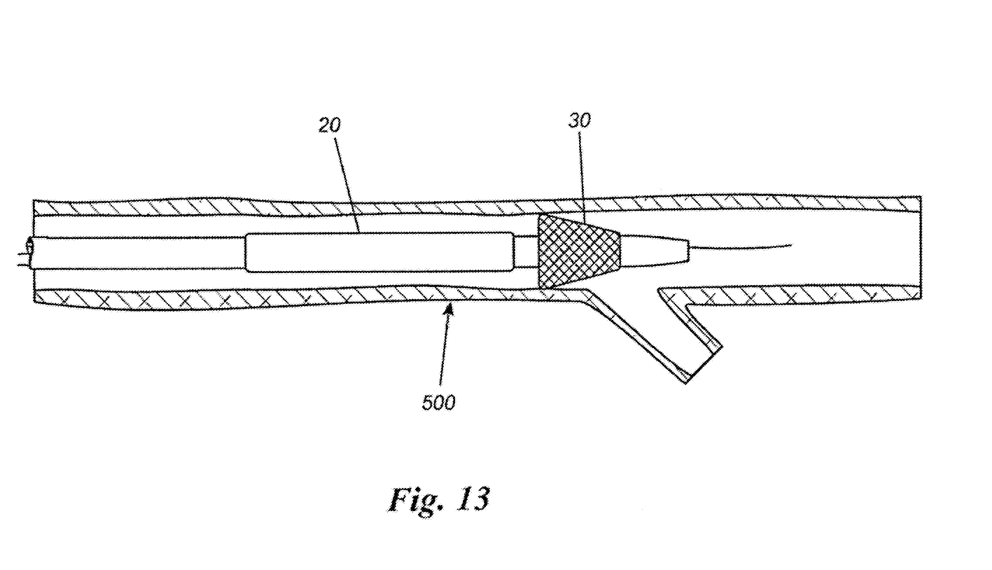

In FIG. 13, the formerly stenosed region can be open, and the neuromodulation balloon 20 has been deflated. The embolic filter 30 remains open to capture any emboli released as the neuromodulation balloon 20 deflates and pulls away from the wall of the vessel 500.

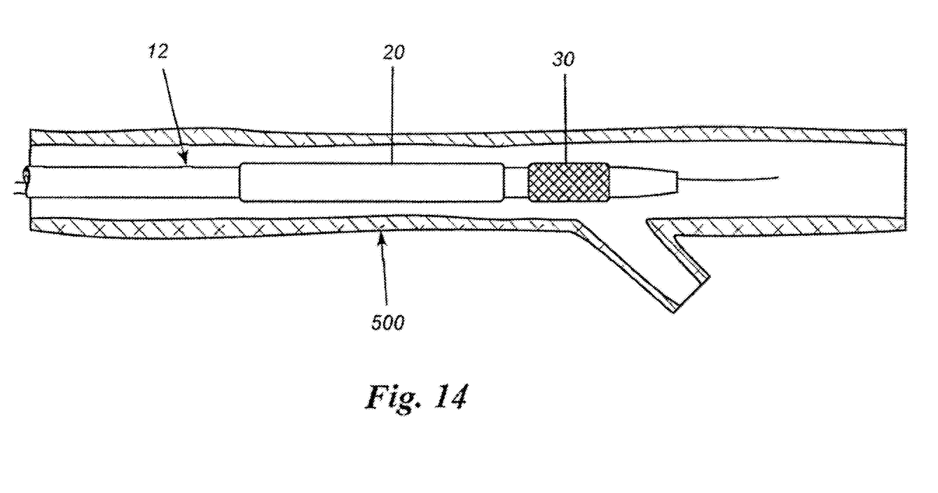

In FIG. 14, the embolic filter 30 can be closed, trapping captured emboli within the filter. The catheter 12 can now be withdrawn from the vessel 500.

### Selected Embodiments of Methods for Neuromodulation

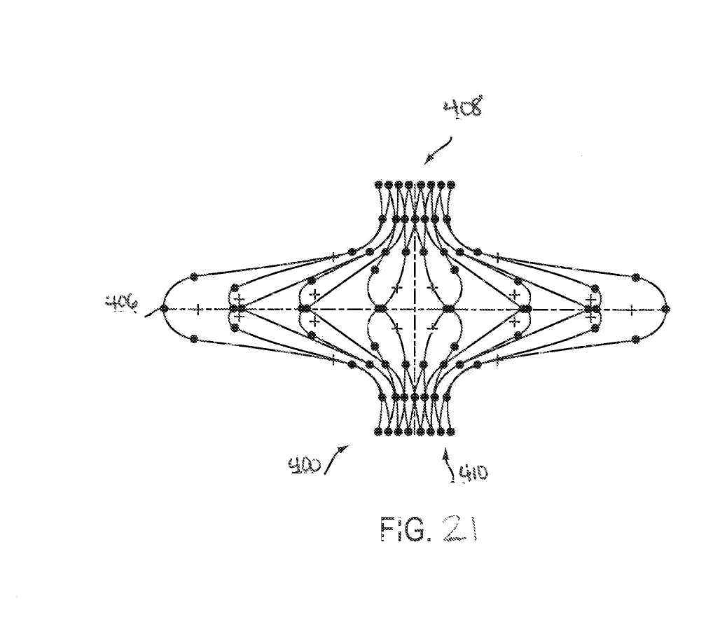

With reference now to FIG. 22, the human renal anatomy includes kidneys K that are supplied with oxygenated blood by renal arteries RA, which are connected to the heart by the abdominal aorta AA. Deoxygenated blood flows from the kidneys to the heart via renal veins RV and the inferior vena cava IVC. FIG. 23 illustrates a portion of the renal anatomy in greater detail. More specifically, the renal anatomy also includes renal nerves RN extending longitudinally along the lengthwise dimension L of renal artery RA generally within the adventitia of the artery. The renal artery RA has smooth muscle cells SMC that surround the arterial circumference and spiral around the angular axis of the artery. The smooth muscle cells of the renal artery accordingly have a lengthwise or longer dimension extending transverse (i.e., non-parallel) to the lengthwise dimension of the renal artery. The misalignment of the lengthwise dimensions of the renal nerves and the smooth muscle cells is defined as “cellular misalignment.”

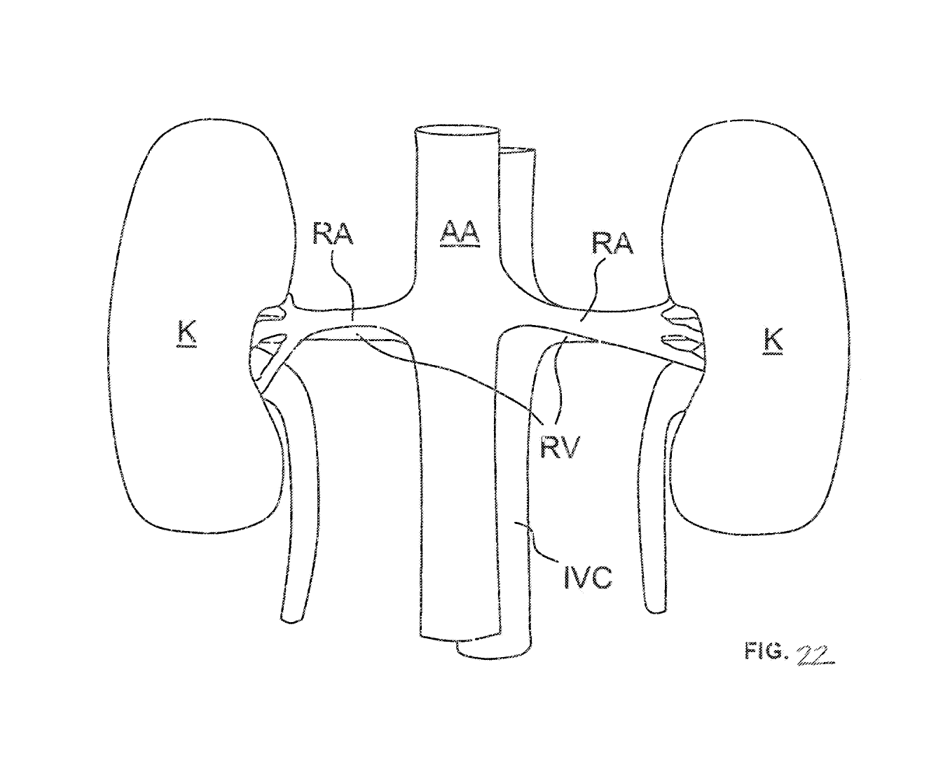

Referring to FIGS. 24A and 24B, the cellular misalignment of the renal nerves and the smooth muscle cells can be exploited to selectively affect renal nerve cells with reduced effect on smooth muscle cells. In one aspect, because larger cells require a lower electric field strength to exceed the cell membrane irreversibility threshold voltage or energy for irreversible electroporation, embodiments of electrodes mounted thereon the neuromodulation device can be configured to align at least a portion of an electric field generated by the electrodes with or near the longer dimensions of the cells to be affected. In particularly aspects, the neuromodulation device can have electrodes configured to create an electrical field aligned with or near the lengthwise dimension L of the renal artery RA to affect renal nerves RN. By aligning an electric field so that the field preferentially aligns with the lengthwise aspect of the cell rather than the diametric or radial aspect of the cell, lower field strengths can be used to affect target neural cells, e.g., to necrose or fuse the target cells, to induce apoptosis, to alter gene expression, to attenuate or block action potentials, to change cytokine up-regulation and/or to induce other suitable processes. It is contemplated that this reduces total energy delivered to the system and mitigates effects on non-target cells in the electric field.

In a further aspect, the lengthwise or longer dimensions of tissues overlying or underlying the target nerve are orthogonal or otherwise off-axis (e.g., transverse) with respect to the longer dimensions of the nerve cells. Thus, in addition to aligning a pulsed electric field with the lengthwise or longer dimensions of the target cells, the pulsed electric field can propagate along the lateral or shorter dimensions of the non-target cells (i.e., such that the pulsed electric field propagates at least partially out of alignment with non-target smooth muscle cells). It is contemplated, as seen in FIGS. 24A and 24B, that the application of a pulsed electric field with propagation lines Li generally aligned with the longitudinal dimension L of the renal artery RA can preferentially cause electroporation (e.g., irreversible electroporation), electrofusion or other neuromodulation in cells of the target renal nerves RN without unduly affecting the non-target arterial smooth muscle cells. In one aspect, the pulsed electric field can propagate in a single plane along the longitudinal axis of the renal artery, or can propagate in the longitudinal direction along any angular segment through a range of about 0° to about 360°.

In another aspect, the neuromodulation device is configured such that a pulsed electric field system can be positioned within and/or in proximity to the wall of the renal artery to selectively propagate an electric field having a longitudinal portion that is aligned to run with the longitudinal dimension of the artery in the region of the renal nerves RN and the smooth muscle cells of the vessel wall so that the interior wall of the artery remains at least substantially intact while the outer nerve cells are destroyed, fused or otherwise affected. It is further contemplated that monitoring elements can be utilized to assess an extent of, e.g., electroporation, induced in renal nerves and/or in smooth muscle cells, as well as to adjust pulsed electric field parameters to achieve a desired effect.

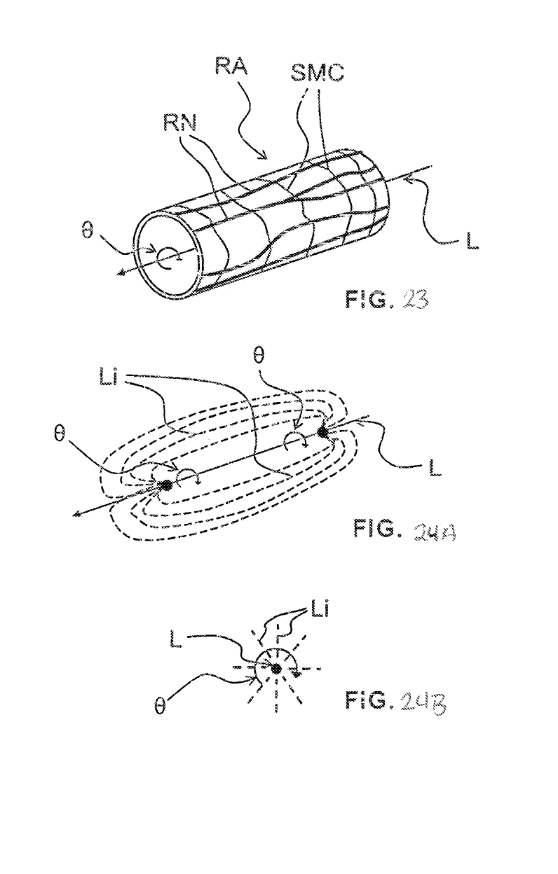

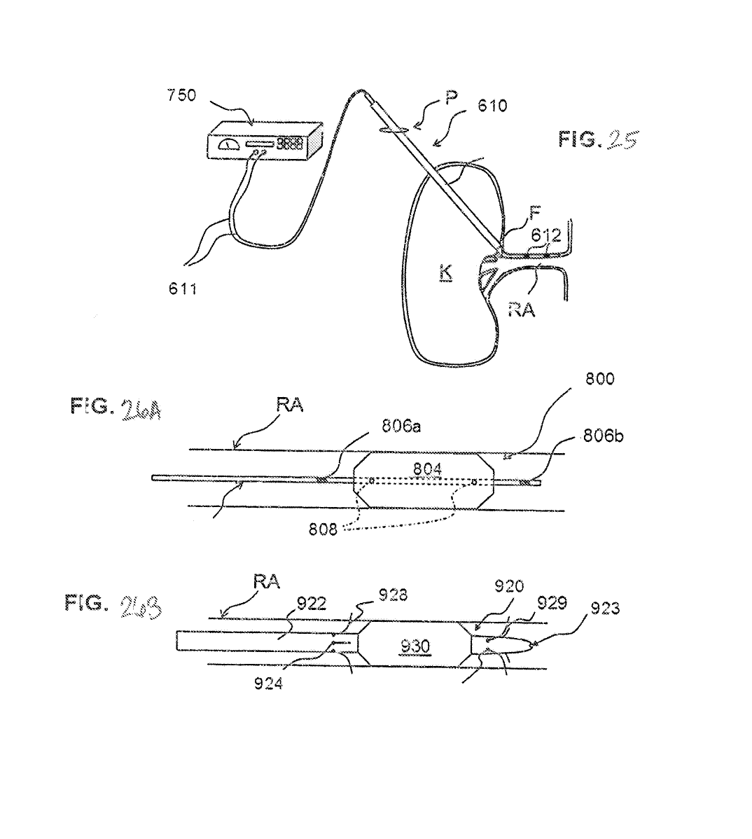

Referring now to FIGS. 25, 26A and 26B, the means for neuromodulation can comprise one or more electrodes that are coupled to the neuromodulation device and are configured to deliver a pulsed electric field to neural fibers of the selected blood vessel to achieve neuromodulation of the desired nerve. While the system, apparatus and method described herein is configured to temporary extravascular placement, it is also contemplated that partially or completely implantable extravascular apparatus additionally or alternatively can be utilized. One exemplary example of a pulsed electric field system is described in U.S. patent application Ser. No. 11/189,563, filed Jul. 25, 2005, which is incorporated herein by reference in its entirety.

An exemplary neuromodulation system is shown in FIG. 25 that comprises an integral embolic filter connected to a laparoscopic or percutaneous pulsed electric field system having a neuromodulation device 610, which is configured for insertion in proximity to the track of desired blood vessel of the patient, such as, for example and without limitation, the renal neural supply along the renal artery or vein or hilum and/or within Gerota's fascia under, e.g., CT or radiographic guidance. In one aspect, at least one electrode 612 can be configured for delivery through the neuromodulation device 610 to a treatment site for delivery of pulsed electric field therapy. In optional aspects, the at least one electrode 612 can be mounted on an expandable portion of a catheter portion of the neuromodulation device and can be electrically coupled to a pulse generator 750 via wires 611. In an alternative aspect, a distal section of the neuromodulation device 610 can have at least one electrode 612, and the neuromodulation device can have an electrical connector to couple the neuromodulation device to the pulse generator 750 for delivering a pulsed electric field therapy to the at least one electrode 612 and hence to the patient.

In this aspect, it is contemplated that the pulsed electric field generator 750 is located external to the patient. The generator, as well as any of the pulsed electric field delivery electrode embodiments described herein, can be utilized with any embodiment of the present invention for delivery of a pulsed electric field with desired field parameters. It should be understood that pulsed electric field delivery electrodes of embodiments described hereinafter can be electrically connected to the generator even though the generator is not explicitly shown or described with each embodiment.

Without limitation, it is contemplated that the at least one electrode 612 can be individual electrodes that are electrically independent of each other, a segmented electrode with commonly connected contacts, or a continuous electrode. A segmented electrode can, for example, be formed by providing a slotted tube fitted onto the electrode, or by electrically connecting a series of individual electrodes. Individual electrodes or groups of electrodes 612 can be configured to provide a bipolar signal. In a further aspect, the electrodes 612 can be dynamically assignable to facilitate monopolar and/or bipolar energy delivery between any of the electrodes and/or between any of the electrodes and an external ground pad. In one aspect, a ground pad can, for example and without limitation, be attached externally to the patient's skin, e.g., to the patient's leg or flank. In FIG. 25, the electrodes 612 comprise a bipolar electrode pair. The neuromodulation device 610 and the coupled electrodes 612 can be similar to the standard needle or trocar-type used clinically for pulsed RF nerve block. Alternatively, the neuromodulation system can comprise a flexible and/or custom-designed probe for the arterial/blood vessel application described herein. Further, it is contemplated that the at least one electrode 612 can comprise a plurality of electrodes that are arranged in a desired array to effect maximal and/or desired degree of nerve denervation upon actuation. In this aspect, it is contemplated that the array can form any desired shape, for example a helical shape, when positioned is a desired location adjacent to the inner wall of the blood vessel.

FIG. 25 shows the insertion of the neuromodulation device with the embolic protection through a percutaneous access site P into proximity with a patient's renal artery RA. In operation, the probe pierces the patient's Gerota's fascia F, and the electrodes 212 are advanced into position through the probe and along the annular space between the patient's artery and fascia. Once properly positioned, the integral embolic filter is deployed and pulsed electric field therapy can be subsequently applied to target neural fibers across the bipolar electrodes 612. Such pulsed electric field therapy can, for example, at least partially denervate the kidney innervated by the target neural fibers through irreversible electroporation of cells of the target neural fibers. The electrodes 612 optionally also can be used to monitor the electroporative effects of the pulsed electric field therapy. After completion of the pulsed electric field therapy, the integral embolic filter can be operatively closed and the apparatus can be removed from the patient to conclude the procedure.

Referring now to FIG. 26A, a further exemplary embodiment of an intravascular pulsed electric field system is shown. Such an exemplary example of a pulsed electric field system is described in U.S. patent application Ser. No. 11/129,765, filed Jan. 25, 2005, which is incorporated herein by reference in its entirety. As shown, this aspect comprises a neuromodulation system comprising a neuromodulation device 800 having a catheter 802 having a centering element 804 (e.g., a balloon, an expandable wire basket, other mechanical expanders, etc.), shaft electrodes 806a and 806b disposed along the shaft of the catheter, and optional radiopaque markers 808 disposed along the shaft of the catheter in the region of the centering element 804. For example and without limitation, the electrodes 806a-b, can be arranged such that the electrode 306a is near a proximal end of the centering element 804 and the electrode 806b is near the distal end of the centering element 804. The electrodes 806 are electrically coupled to the pulse generator 750 (see FIG. 4), which is disposed external to the patient, for delivery of the pulsed electric field therapy.

In one aspect, it is contemplated that the centering element 804 can comprise an impedance-altering element that alters the impedance between electrodes 806a and 806b during the pulsed electric field therapy. In this aspect, the alteration of impedance provides for better direction of the pulsed electric field therapy across the vessel wall, which can reduce an applied voltage required to achieve desired renal neuromodulation. Such an exemplary example of a pulsed electric field system using an impedance-altering element is described in U.S. patent application Ser. No. 11/266,993, filed Nov. 4, 2005, which is incorporated herein by reference in its entirety. Here, when the centering element 804 comprises an conventional inflatable balloon, the balloon can serve as both the centering element for the electrodes 806 and as an impedance-altering electrical insulator for directing an electric field delivered across the electrodes, e.g., for directing the electric field into or across the vessel wall for modulation of target neural fibers. Electrical insulation provided by the element 804 can reduce the magnitude of applied voltage or other parameters of the pulsed electric field necessary to achieve desired field strength at the target fibers.

In one aspect, it is contemplated that the electrodes 806 can be individual electrodes (i.e., independent contacts), a segmented electrode with commonly connected contacts, or a single continuous electrode. Furthermore, the electrodes 806 can be configured to provide a bipolar signal, or the electrodes 806 can be used together or individually in conjunction with a separate patient ground pad for monopolar use. As an alternative or in addition to placement of the electrodes 806 along the central shaft of catheter 802, as in FIG. 22A, the electrodes 806 can be attached to the centering element 804 such that they contact the wall of the vessel, such as the renal artery RA.

In a further aspect, the electrodes can, for example, be affixed to the inside surface, outside surface or at least partially embedded within the wall of the centering element. The electrodes optionally can be used to monitor the effects of pulsed electric field therapy, as described hereinafter. As it can be desirable to reduce or minimize physical contact between the pulsed electric field-delivery electrodes and the vessel wall during delivery of pulsed electric field therapy, e.g., to reduce the potential for injuring the wall, the electrodes 806 can, for example, comprise a first set of electrodes attached to the shaft of the catheter for delivering the pulsed electric field therapy, and the device can further include a second set of electrodes optionally attached to the centering element 804 for monitoring the effects of pulsed electric field therapy delivered via the electrodes 806. Further, it is contemplated that the electrodes 806 can comprise a plurality of electrodes arranged in a desired array to effect maximal and/or desired degree of nerve denervation upon actuation. In this aspect, it is contemplated that the array can form any desired shape, for example a helical shape, when positioned is a desired location adjacent to the inner wall of the blood vessel.

In operation, the catheter 802 with a coupled integral embolic filter can be delivered to the renal artery RA as shown, or it can be delivered to a renal vein or to any other vessel in proximity to neural tissue contributing to renal function, in a low profile delivery configuration, for example, through a guide catheter. Once positioned within the renal vasculature, the integral embolic filter can be expanded to operational position and, subsequently, the optional centering element 804 can be expanded into contact with an interior wall of the vessel. Next, a pulsed electric field can be generated by the pulsed electric field generator 750, transferred through the catheter 802 to the electrodes 806, and delivered via the electrodes or array of electrodes 806 across the wall of the artery. As described above, the pulsed electric field therapy modulates the activity along neural fibers that contribute to renal function, e.g., at least partially denervates the kidney innervated by the neural fibers. In various optional aspects, this therapy can be achieved, for example, via irreversible electroporation, electrofusion and/or inducement of apoptosis in the nerve cells. In many applications, it is contemplated that the electrodes can be arranged so that the pulsed electric field is aligned with the longitudinal dimension of the renal artery to facilitate modulation of renal nerves with little effect on non-target smooth muscle cells or other cells.

Optionally, intra to extravascular pulsed electric field systems can be provided having at least one electrode that is delivered to an intravascular position, then at least partially passed through/across the inner vessel wall to an extravascular position prior to delivery of pulsed electric field PEF therapy. In this aspect, the extravascular positioning of the at least one electrode can place the electrode in closer proximity to target neural fibers during the pulsed electric field therapy compared to fully intravascular positioning of the electrode. Such an exemplary example of a extravascular pulsed electric field system is described in U.S. patent application Ser. No. 11/324, filed Dec. 29, 2005, which is incorporated herein by reference in its entirety.

Referring to FIG. 26B, one exemplary aspect of an intra to extravascular pulsed electric field system is shown. In one aspect, the exemplary intra to extravascular pulsed electric field system 920 comprises a catheter 922 comprising (a) a plurality of proximal electrode lumens terminating at proximal side ports 924, (b) a plurality of distal electrode lumens terminating at distal side ports 926, and (c) a guidewire lumen 923. In one aspect, the catheter 922 preferably comprises an equal number of proximal and distal electrode lumens and side ports. The system 920 can also comprise proximal needle electrodes 928 that can be configured to be advanced through the proximal electrode lumens and the proximal side ports 924, as well as distal needle electrodes 929 that can be configured to be advanced through the distal electrode lumens and the distal side ports 926.

As shown, catheter 922 comprises an optional expandable centering element 930, which can comprise an inflatable balloon or an expandable basket or cage. In operation, the integral embolic filter can be expanded to operational position and, subsequently, the centering element 930 can be expanded prior to deployment of the needle electrodes 928 and 929 in order to center the catheter 922 within the patient's vessel (e.g., within renal artery RA). Centering the catheter 922 is expected to facilitate delivery of all needle electrodes to desired depths within/external to the patient's vessel (e.g., to deliver all of the needle electrodes approximately to the same depth). In FIG. 26B, the illustrated centering element 330 is positioned between the proximal side ports 924 and the distal side ports 926, i.e., between the delivery positions of the proximal and distal electrodes. However, it should be understood that centering element 930 additionally or alternatively can be positioned at a different location or at multiple locations along the length of the catheter 922 (e.g., at a location proximal of the side ports 924 and/or at a location distal of the side ports 926).

Exemplarily, it is contemplated that the catheter 922 can be advanced to a treatment site within the patient's vasculature (e.g., to a treatment site within the patient's renal artery RA) over a guidewire (not shown) via the lumen 923. During intravascular delivery, the electrodes 928 and 929 can be positioned such that their non-insulated and sharpened distal regions are positioned within the proximal and distal lumens, respectively. Once positioned at a treatment site, a medical practitioner can advance the electrodes via their proximal regions that are located external to the patient. Such advancement causes the distal regions of the electrodes 928 and 929 to exit side ports 924 and 926, respectively, and pierce the wall of the patient's vasculature such that the electrodes are positioned extravascularly.

In one aspect, the proximal electrodes 928 can be connected to pulsed electric field generator 750 as active electrodes and the distal electrodes 929 can serve as return electrodes. In this example, the proximal and distal electrodes form bipolar electrode pairs that align pulsed electric field therapy with a longitudinal axis or direction of the patient's vasculature. As will be apparent to one skilled in the art, the distal electrodes 929 alternatively can comprise the active electrodes and the proximal electrodes 928 can comprise the return electrodes. Furthermore, the proximal and/or the distal electrodes can comprise both active and return electrodes. In is contemplated that any combination of active and distal electrodes can be utilized, as desired.

When the electrodes 928 and 929 are connected to pulsed electric field generator 750 and are positioned extravascularly, and with the integral embolic device expanded and the centering element 930 optionally expanded, pulsed electric field therapy can proceed to achieve desired neuromodulation. After completion of the pulsed electric field therapy, the electrodes can be retracted within the proximal and distal lumens, and centering element 930 can be collapsed for retrieval. Subsequently, the integral embolic device can be collapsed and the system can be removed from the patient to complete the procedure. Additionally or alternatively, the system can be repositioned to provide pulsed electric field therapy at another treatment site, for example, to provide bilateral renal neuromodulation.

In one aspect, it is contemplated that pulsed electric field therapy, as well as other methods and apparatus of the present invention for neuromodulation (e.g., stimulation electric fields, localized drug delivery, high frequency ultrasound, thermal techniques, etc.), whether delivered extravascularly, intravascularly, intra to extravascularly or a combination thereof, can, for example, effectuate irreversible electroporation or electrofusion, necrosis and/or inducement of apoptosis, alteration of gene expression, action potential blockade or attenuation, changes in cytokine up-regulation and other conditions in target neural fibers. In some patients, when such neuromodulatory methods and apparatus are applied to renal nerves and/or other neural fibers that contribute to renal neural functions, neuromodulatory effects induced by the neuromodulation can result in increased urine output, decreased plasma renin levels, decreased tissue (e.g., kidney) and/or urine catecholamines (e.g., norepinephrine), increased urinary sodium excretion, and/or controlled blood pressure. Furthermore, it is contemplated that these or other changes might prevent or treat congestive heart failure, hypertension, acute myocardial infarction, end-stage renal disease, contrast nephropathy, other renal system diseases, and/or other renal or cardio-renal anomalies for a period of months, potentially up to six months or more. This time period can be sufficient to allow the body to heal; for example, this period can reduce the risk of congestive heart failure onset after an acute myocardial infarction, thereby alleviating a need for subsequent re-treatment. Alternatively, as symptoms reoccur, or at regularly scheduled intervals, the patient can return to the physician for a repeat therapy without worry of the incidence of an embolic event in the course of the applied therapy.

The methods and apparatus described herein could be used to modulate efferent or afferent nerve signals, as well as combinations of efferent and afferent nerve signals. In one aspect, neuromodulation can be achieved without completely physically severing, i.e., without fully cutting, the target neural fibers. However, it should be understood that such neuromodulation can functionally sever the neural fibers, even though the fibers cannot be completely physically severed. Apparatus and methods described herein illustratively are configured for percutaneous use. Such percutaneous use can be endoluminal, laparoscopic, a combination thereof, etc.

Optionally, the apparatus described above with respect to FIGS. 25, 26A and 26B can be used to quantify the efficacy, extent or cell selectivity of pulsed electric field therapy to monitor and/or control the therapy. When a pulsed electric field initiates electroporation, the impedance of the electroporated tissue begins to decrease and the conductivity of the tissue begins to increase. If the electroporation is reversible, the tissue electrical parameters will return or approximate baseline values upon cessation of the pulsed electric field. However, if the electroporation is irreversible, the changes in tissue parameters will persist after termination of the pulsed electric field. These phenomena can be utilized to monitor both the onset and the effects of pulsed electric field therapy. For example, electroporation can be monitored directly using, for example, conductivity measurements or impedance measurements, such as Electrical Impedance Tomography and/or other electrical impedance/conductivity measurements like an electrical impedance or conductivity index. Such electroporation monitoring data optionally can be used in one or more feedback loops to control delivery of pulsed electric field therapy.

In a further aspect, the system can comprise monitoring electrodes positioned in proximity to the targeted tissue. One will appreciate that while FIGS. 25, 26A and 26B illustratively comprise bipolar apparatus, it should be understood that monopolar apparatus alternatively can be utilized. For example, an active monopolar electrode can be positioned intravascularly, extravascularly or intra-to-extravascularly in proximity to target neural fibers that contribute to renal function. A return electrode ground pad can be attached to the exterior of the patient. Finally, pulsed electric field therapy can be delivered between to the in vivo monopolar electrode and the ground pad to effectuate desired renal neuromodulation. Monopolar apparatus additionally can be utilized for bilateral renal neuromodulation.

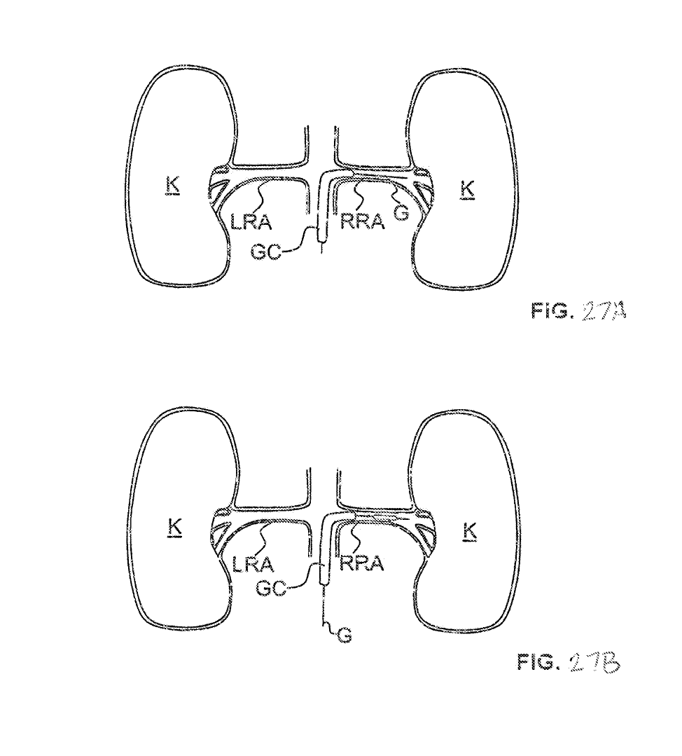

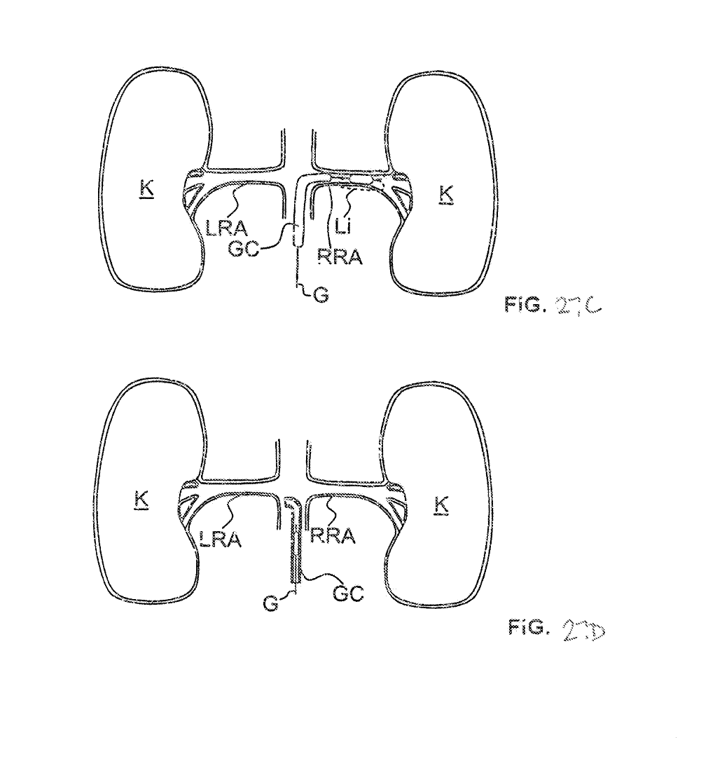

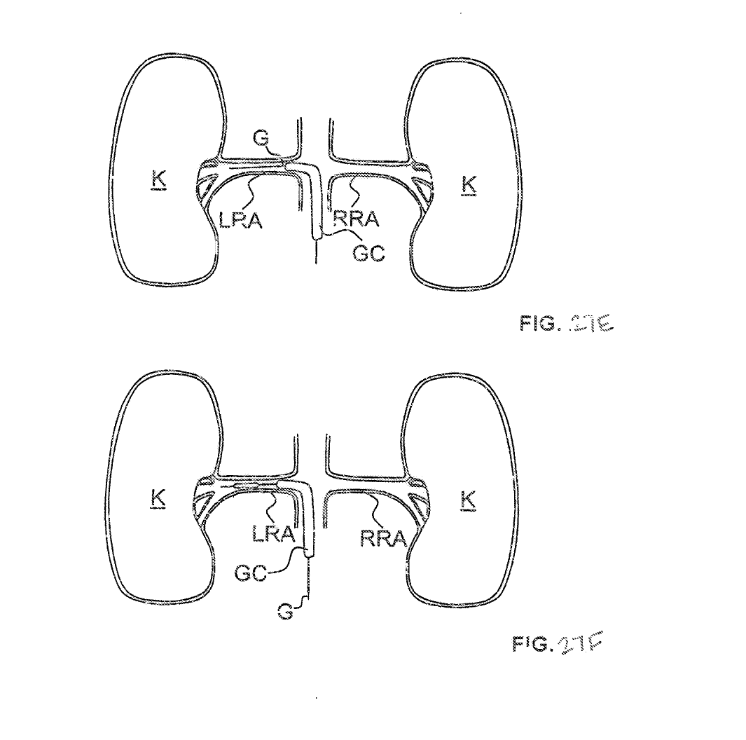

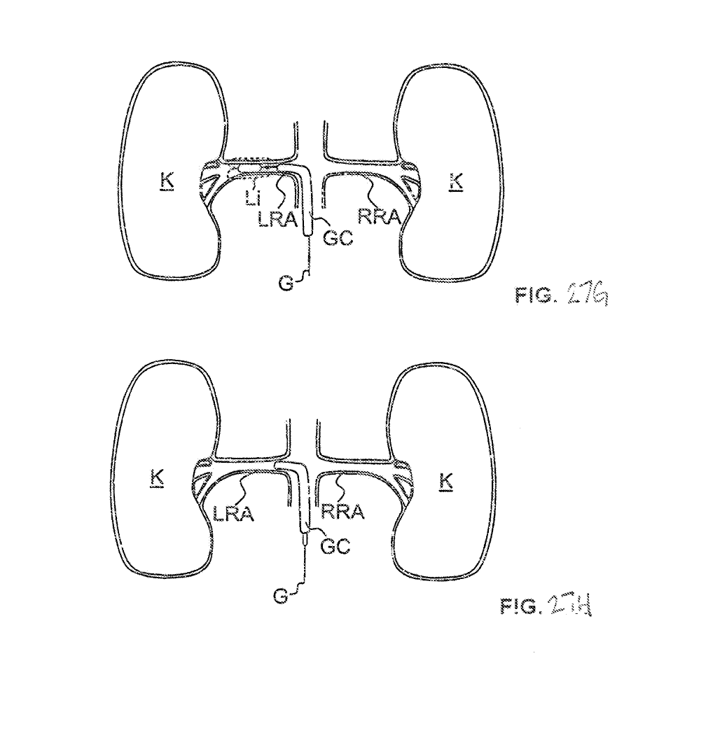

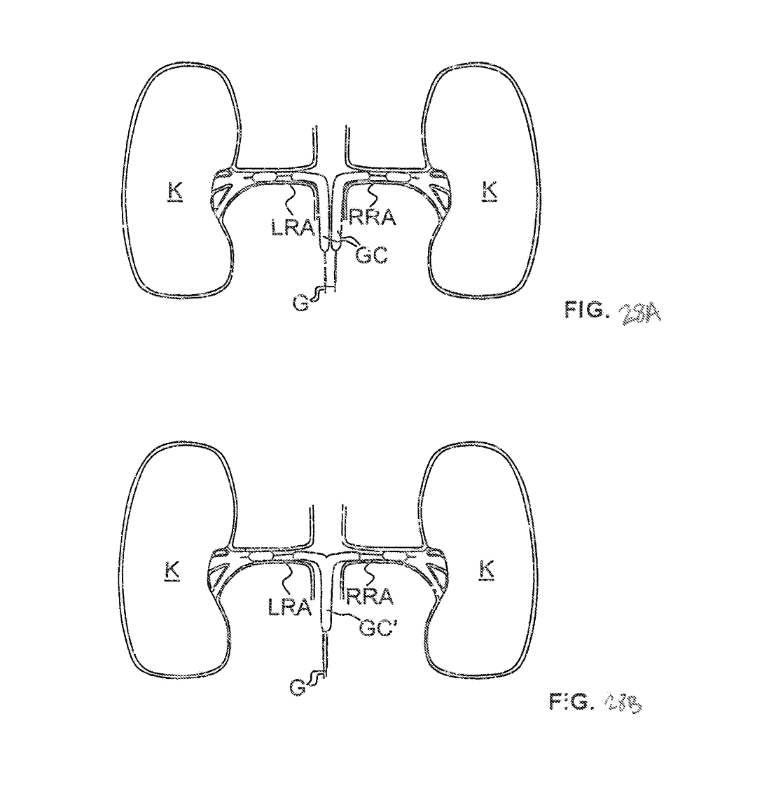

Referring to FIGS. 27A-23H, a method for bilateral renal neuromodulation utilizing the intravascular apparatus of FIG. 26A is illustrated. However, it should be understood that such bilateral neuromodulation alternatively can be achieved utilizing the extravascular apparatus of FIG. 25, utilizing the intra-to-extravascular apparatus of FIG. 27B, or utilizing any alternative intravascular apparatus, extravascular apparatus, intra-to-extravascular apparatus (including monopolar apparatus) or combination thereof.

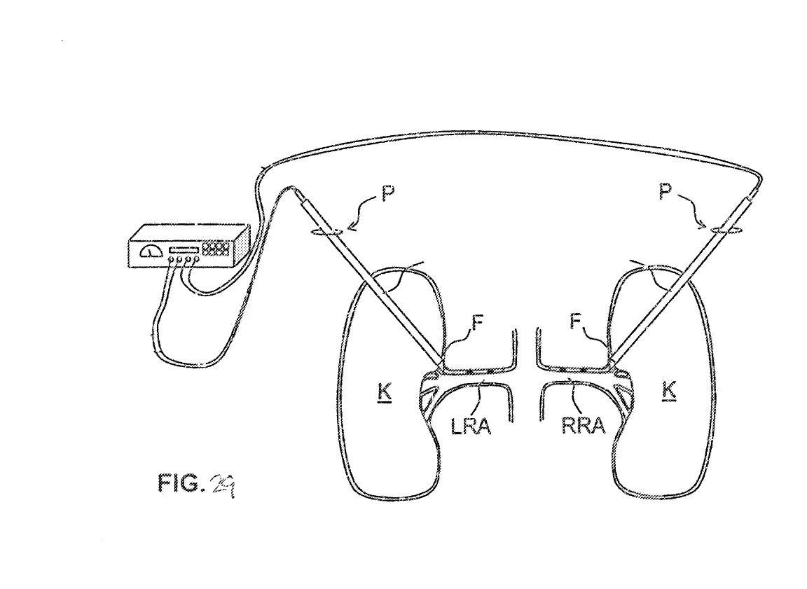

As seen in FIGS. 27A and 27E, a guide catheter GC and a guidewire G can be advanced into position within, or in proximity to, either the patient's left renal artery LRA or right renal artery RRA. In FIG. 26A, the guidewire illustratively has been positioned in the right renal artery RRA, but it should be understood that the order of bilateral renal neuromodulation illustrated in FIGS. 27A-27H alternatively can be reversed. Additionally or alternatively, bilateral renal neuromodulation can be performed concurrently on both right and left neural fibers that contribute to renal function, as in FIGS. 28A-30, rather than sequentially, as in FIG. 27A-27H.

In operation, with the guidewire and the guide catheter positioned in the right renal artery, the catheter of the apparatus can be advanced over the guidewire and through the guide catheter into position within the artery. As seen in FIG. 27B, the optional centering element of the catheter and the integral embolic filter are in a reduced delivery configuration during delivery of the catheter to the renal artery. In FIG. 27C, once the catheter is properly positioned for denervation therapy, first the integral embolic filter and subsequently the centering element optionally can be expanded into contact with the vessel wall, and the guidewire G can be retracted from the treatment zone, e.g., can be removed from the patient or can be positioned more proximally within the patient's aorta.

As one will appreciate, expansion of element can center the electrodes within the vessel and/or can alter impedance between the electrodes. With apparatus positioned and deployed as desired, denervation therapy can be delivered in a bipolar fashion across the electrodes to achieve renal neuromodulation in neural fibers that contribute to right renal function, e.g., to at least partially achieve renal denervation of the right kidney. As illustrated by propagation lines Li, the pulsed electric field can be aligned with a longitudinal dimension of the renal artery RA and can pass across the vessel wall. The alignment and propagation path of the pulsed electric field is expected to preferentially modulate cells of the target renal nerves without unduly affecting non-target arterial smooth muscle cells.

Referring to FIG. 27D, after completion of the denervation therapy, the centering element and then the integral embolic filter can be collapsed back to the reduced delivery profile, and the catheter can be retracted from the right renal artery RRA, for example, to a position in the guide catheter GC within the patient's abdominal aorta. Likewise, the guide catheter GC can be retracted to a position within the patient's aorta. The retracted guide catheter can be repositioned, e.g., rotated, such that its distal outlet is generally aligned with the left renal artery LRA. The guidewire G then can be re-advanced through the catheter and the guide catheter GC to a position within the left renal artery LRA, as shown in FIG. 27E (as will be apparent, the order of advancement of the guidewire and the guide catheter optionally can be reversed when accessing either renal artery).

Next, the catheter can be re-advanced over the guidewire and through the guide catheter into position within the left renal artery, as shown in FIG. 27F. In FIG. 27G, once the catheter is properly positioned for denervation therapy, first the integral embolic filter and subsequently the centering element optionally can be expanded into contact with the vessel wall, and the guidewire G can be retracted to a position proximal of the treatment site. Denervation therapy then can be delivered in a bipolar fashion across the electrodes, for example, along propagation lines Li, to achieve renal neuromodulation in neural fibers that contribute to left renal function, e.g., to at least partially achieve renal denervation of the left kidney. As seen in FIG. 27H, after completion of the bilateral denervation therapy, the centering element and then the integral embolic filter can be collapsed back to the reduced delivery profile, and the catheter, as well as the guidewire G and the guide catheter GC, can be removed from the patient to complete the bilateral renal neuromodulation procedure.

FIGS. 28A and 28B illustrate optional aspects for performing concurrent bilateral renal neuromodulation. In the embodiment of FIG. 28A, apparatus comprises dual denervation therapy catheters with integral embolic filters, as well as dual guidewires G and guide catheters GC. One catheter is positioned within the right renal artery RRA, and the other catheter is positioned within the left renal artery LRA. With catheters positioned in both the right and left renal arteries, denervation therapy can be delivered concurrently by the catheters to achieve concurrent bilateral renal neuromodulation, illustratively via an intravascular approach.

In a further aspect illustrated in FIG. 29, methods and apparatus for concurrent bilateral renal neuromodulation are shown. In this aspect, the extravascular apparatus comprises dual neuromodulation devices. The electrodes are positioned in the vicinity of both the left renal artery LRA and the right renal artery RRA. Denervation therapy can be delivered concurrently by the electrodes to achieve concurrent bilateral renal neuromodulation, illustratively via an extravascular approach.

Optionally, and in further aspects, denervation methods and apparatus for achieving the desired degree of neuromodulation can further comprise one or more of: denervation via localized drug delivery (such as by a drug pump or infusion catheter), denervation via use of a stimulation electric field, and the like. Such exemplary examples are described in U.S. patent application Ser. No. 10/408,665, filed Apr. 8, 2003, and in U.S. Pat. No. 6,978,174, which are incorporated herein by reference in its entirety.

With respect to FIG. 30, denervation methods and apparatus for achieving bilateral renal neuromodulation via localized drug delivery is shown. In this aspect, drug reservoir 1002, illustratively an implantable drug pump, has been implanted within the patient. Drug delivery catheters 1000a and 1000b are connected to the drug reservoir and extend to the vicinity of the right renal artery RRA and the left renal artery LRA, respectively, for delivery of one or more neuromodulatory agents or drugs capable of modulating neural fibers that contribute renal function. Delivering the agent(s) through catheters 1000a and 1000b can achieve bilateral renal neuromodulation. Such drug delivery through catheters can be conducted concurrently or sequentially, as well as continuously or intermittently, as desired, in order to provide concurrent or sequential, continuous or intermittent, renal neuromodulation, respectively. Of course, the integral embolic filter can be operative deployed to prevent any undesired embolic event that could occur as a result of the catheter deployment or the denervation therapy procedure.

Optionally it is contemplated that the catheters 1000a and 1000b can be positioned temporarily at the desired location for acute delivery of the neuromodulatory agent(s) from an external drug reservoir, such as a syringe. Such temporary positioning can comprise, for example, intravascular, extravascular and/or intra-to-extravascular placement of the catheters. In another alternative embodiment, the drug reservoir 1002 can be replaced with an implantable neurostimulator or a pacemaker-type device, and catheters 1000 can be replaced with electrical leads coupled to the neurostimulator for delivery of an electric field, such as a pulsed electric field or a stimulation electric field, to the target neural fibers. In yet another alternative embodiment, electrical techniques can be combined with delivery of neuromodulatory agent(s) to achieve desired bilateral renal neuromodulation. Of course, the integral embolic filter can be operative deployed to prevent any undesired embolic event that could occur as a result of the catheter deployment or the denervation therapy procedure.

In yet another aspect of the present invention, it is contemplated that the system and methods described herein can be configured to treat other diseases resulting from hyperactivity of sympathetic and parasympathetic nerves comprise delivery of neuoromodulating agents for the chemical or neuromodulating devernation of arteries. While these systems and methods have been described in detail with respect to application to the renal renal arteries, one skilled in the art will appreciate that other vascular beds can benefit from these methods. In one aspect, for example and without limitation, devernation of the carotid carotid artery can be used to treat patients with carotid sinus syndrome (CSS), which is a condition that leads to dizziness and syncope but can be rectified by carotid adventitial devernation.

In a further aspect, it is contemplated that the methods and systems disclosed herein may be applied to satisfy clinical needs related to treating cardiac, metabolic, and pulmonary diseases associated, at least in part, with enhanced chemoreflex (e.g. high chemosensor sensitivity or high chemosensor activity) and related sympathetic activation. In one aspect, the system and methods described herein can be configured to treat other diseases resulting from hyperactivity of sympathetic and parasympathetic. Enhanced peripheral and central chemoreflex is implicated in several pathologies including hypertension, cardiac tachyarrhythmias, sleep apnea, dyspnea, chronic obstructive pulmonary disease (COPD), diabetes and insulin resistance, and CHF. Central sympathetic nervous system activation is common to all these progressive and debilitating diseases. Peripheral chemoreflex may be modulated, for example, by modulating carotid body activity by ablating a carotid body or afferent nerves emerging from the carotid body. Thus, in a further aspect, it is contemplated that the system and methods described herein can be configured to restore desired nervous activity by reducing or removing carotid body input into the central nervous system.

Thus, implementations of the foregoing provide various desirable features. For instance, the present disclosure permits the placement of the embolic filter very close to the means for treating the stenosis. This has the effect of minimizing the “landing area” of the filter and also permits the protection of side branches.

The present invention can thus be embodied in other specific forms without departing from its spirit or essential characteristics. The described aspects are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A percutaneous transluminal neuromodulation device, comprising:
an elongated catheter having proximal and distal ends and an outer side wall;
a neuromodulation device attached to the catheter adjacent the distal end thereof;
a filter attached to the elongated catheter between the neuromodulation device and the distal end of the catheter, the filter being collapsible for insertion of the distal end of the catheter into a blood vessel, and the filter being expandable to an expanded position to capture emboli released into a bloodstream by operation of the neuromodulation device, wherein the filter comprises:
a movable ring portion movably attached to the catheter;
a fixed ring portion immovably attached to the catheter such that the movable ring portion is movable relative to the fixed ring portion, wherein the movable ring portion is distal to the fixed ring portion;
a braided filter scaffolding that is formed of a shape memory material that urges the braided filter scaffolding into a base line closed or collapsed position, a distal end of the braided filter scaffolding is coupled to the movable ring portion and a proximal end of the braided filter scaffolding is coupled to the fixed ring portion; and
a filter mesh overlying a portion of the braided filter scaffolding;
wherein the catheter further comprises a lumen and a port in communication with the lumen, the port comprising an aperture in the outer side wall of the catheter located distal to the fixed ring portion and proximal to the movable ring portion, and the lumen extending from a location proximate the proximal end of the catheter to the port; and
an actuator wire having proximal and distal ends, the actuator wire extending through the lumen of the catheter, and the distal end of the actuator wire exiting the lumen of the catheter through the port, the distal end of the actuator wire being attached to the movable ring portion;
wherein, when the filter is in the collapsed position, pulling on the proximal end of the wire exerts a force on the movable ring portion in the proximal direction that moves the movable ring portion toward the fixed ring portion and causes the braided filter scaffolding to bow outward to expand the filter to the expanded position;
wherein, when the filter is in the expanded position, releasing tension on the wire permits the shape memory of the braided filter scaffolding to return the braided filter scaffolding to the base line closed or collapsed position, collapsing the filter.

2. The percutaneous transluminal neuromodulation device of claim 1, wherein the neuromodulation device comprises a neuromodulation balloon.

3. The percutaneous transluminal neuromodulation device of claim 1, wherein the neuromodulation device comprises a neuromodulation stent.

4. The percutaneous transluminal neuromodulation device of claim 1, wherein the neuromodulation device comprises an energy delivery device.

5. The percutaneous transluminal neuromodulation device of claim 1, wherein the shape memory material comprises Nitinol or Cobalt-Chromium.

6. The percutaneous transluminal neuromodulation device of claim 1, wherein filter mesh overlies a distal portion of the braided filter scaffolding, and wherein, in the expanded position, the braided filter scaffolding bows outward, radially expanding the filter mesh.

7. The percutaneous transluminal neuromodulation device of claim 1, wherein the filter mesh extends beyond the braided filter scaffolding in a longitudinal direction relative to the longitudinal axis of the catheter, such that a sac is formed to retain embolic particles when the filter is in the collapsed position.

8. The percutaneous transluminal neuromodulation device of claim 1, wherein the braided filter scaffolding comprises, metal wires, polymer wires and the like.

9. The percutaneous transluminal neuromodulation device of claim 8, wherein the braided filter scaffolding is formed from a wire braid comprising from between about 12 to about 16 wires.

10. The percutaneous transluminal neuromodulation device of claim 9, wherein the wires comprising the braided filter scaffolding can have a rounded profile in cross-section.

11. The percutaneous transluminal neuromodulation device of claim 1, wherein the wires comprising the braided filter scaffolding can have a flat profile in cross-section.

12. The percutaneous transluminal neuromodulation device of claim 1, wherein a braiding angle between the wires of the braided filter scaffolding and a longitudinal axis of the braided filter scaffolding is a multiple between about 1.5× and 4× of the angle between the wire and the central axis when the wire is in the base line closed or collapsed position.

13. The percutaneous transluminal neuromodulation device of claim 1, wherein a braiding angle between the wires of the braided filter scaffolding and a longitudinal axis of the braided filter scaffolding is a multiple between about 1.7× and 3× of the angle between the wire and the central axis when the wire is in the base line closed or collapsed position.

14. The percutaneous transluminal neuromodulation device of claim 1, wherein a braiding angle between the wires of the braided filter scaffolding and a longitudinal axis of the braided filter scaffolding is a multiple of about double (2×) of the angle between the wire and the central axis when the wire is in the base line closed or collapsed position.

15. The percutaneous transluminal neuromodulation device of claim 1, wherein a braiding angle between the wires of the braided filter scaffolding and a longitudinal axis of the braided filter scaffolding is a about 150 degrees.

16. The percutaneous transluminal neuromodulation device of claim 1, wherein the braided filter scaffolding forms a relatively wide mesh when opened in order to allow blood flow into the filter membrane.

17. A percutaneous transluminal neuromodulation device, comprising:
an elongated catheter having proximal and distal ends;
a neuromodulation device attached to the catheter adjacent the distal end thereof;
a filter attached to the elongated catheter between the neuromodulation device and the distal end of the catheter, the filter being collapsible for insertion and removal of the distal end of the catheter into a blood vessel, and the filter being expandable to an expanded position to capture emboli released into a bloodstream by operation of the neuromodulation device, wherein the filter comprises:
a movable ring portion movably attached to the catheter;
a fixed ring portion immovably attached to the catheter such that the movable ring portion is movable relative to the fixed ring portion;
a braided filter scaffolding that is formed of a shape memory material that urges the braided filter scaffolding into a base line closed or collapsed position, a distal end of the braided filter scaffolding is coupled to the movable ring portion and a proximal end of the braided filter scaffolding is coupled to the fixed ring portion; and
a filter mesh overlying a portion of the braided filter scaffolding;
wherein the catheter further comprises a lumen extending from a location proximate the proximal end of the catheter, to a location distal to the neuromodulation device; and
an actuator wire having proximal and distal ends, the actuator wire extending through the lumen of the catheter, the proximal end of the actuator wire extending to a location proximate the proximal end of the catheter and the distal end of the actuator wire exiting the lumen through the side wall of the catheter at the location distal to the neuromodulation device, the distal end of the actuator wire being attached to the movable ring portion;
wherein when the filter is in a collapsed condition, manipulating the proximal end of the wire exerts a force on the movable ring portion that moves the movable ring portion toward the fixed ring portion and causes the braided filter scaffolding to bow outward to the expanded position.

18. The percutaneous transluminal neuromodulation device of claim 17, wherein the movable ring portion is the distal ring portion.

19. The percutaneous transluminal neuromodulation device of claim 18, wherein the distal end of the actuator wire exits the lumen through the catheter side wall at a location distal to the proximal ring portion.

20. The percutaneous transluminal neuromodulation device of claim 19, wherein the distal end of the actuator wire is operatively connected to the distal ring portion.

21. The percutaneous transluminal neuromodulation device of claim 20, wherein pulling on the proximal end of the actuator wire draws the distal ring portion toward the fixed proximal ring portion.

22. The percutaneous transluminal neuromodulation device of claim 17, wherein the neuromodulation device comprises a neuromodulation balloon.

23. The percutaneous transluminal neuromodulation device of claim 17, wherein the neuromodulation device comprises a neuromodulation stent.

24. The percutaneous transluminal neuromodulation device of claim 17, wherein the neuromodulation device comprises an energy delivery device.

25. The percutaneous transluminal neuromodulation device of claim 17, wherein the shape memory material comprises Nitinol or Cobalt-Chromium.

26. The percutaneous transluminal neuromodulation device of claim 17, wherein filter mesh overlies a distal portion of the braided filter scaffolding, and wherein, in the expanded position, the ribs bow outward, radially expanding the filter mesh.

27. The percutaneous transluminal neuromodulation device of claim 17, wherein the filter mesh extends beyond the braided filter scaffolding in a longitudinal direction relative to the longitudinal axis of the catheter, such that a sac is formed to retain embolic particles when the filter is in the collapsed position.

28. The percutaneous transluminal neuromodulation device of claim 17, wherein the braided filter scaffolding comprises, metal wires, polymer wires and the like.

29. The percutaneous transluminal neuromodulation device of claim 17, wherein the braided filter scaffolding is formed from a wire braid comprising from between about 12 to about 16 wires.

30. The percutaneous transluminal neuromodulation device of claim 17, wherein a braiding angle between the wires of the braided filter scaffolding and a longitudinal axis of the braided filter scaffolding is a multiple between about 1.5× and 4× of the angle between the wire and the central axis when the wire is in the base line closed or collapsed position.
